SEQUENCE LISTING (6 sequences)

SEQ ID NO: 1 - DNA, 37 bp, Artificial Sequence
  Description of Artificial Sequence Synthetic oligonucleotide
tgctatatccttatgctgcttggggggatccagtact

SEQ ID NO: 2 - DNA, 37 bp, Artificial Sequence
  Description of Artificial Sequence Synthetic oligonucleotide
ctgcaggatgttctcatgcttggggggatccagtact

SEQ ID NO: 3 - DNA, 20 bp, Artificial Sequence
  Description of Artificial Sequence Synthetic primer
aaaatgcccccaacagagcc

SEQ ID NO: 4 - DNA, 24 bp, Artificial Sequence
  Description of Artificial Sequence Synthetic primer
ccaccagcaaatacacaacagcac

SEQ ID NO: 5 - DNA, 20 bp, Artificial Sequence
  Description of Artificial Sequence Synthetic primer
ccacactgtgcccatctacg

SEQ ID NO: 6 - DNA, 21 bp, Artificial Sequence
  Description of Artificial Sequence Synthetic primer
gatcttcatggtgctaggagc

(19) United States
(12) Patent Application Publication
(10) Pub. No.: US 2010/0240732 A1
(43) Pub. Date: Sep. 23, 2010

(54) APTAMER-TARGETED SIRNA TO PREVENT ATTENUATION OR SUPPRESSION OF A T CELL FUNCTION

(75) Inventor: Eli Gilboa, Coral Gables, FL (US)

Correspondence Address: NOVAK DRUCE + QUIGG LLP (WPB), 525 Okeechobee Blvd, 15th Floor, City Place Tower, West Palm Beach, FL, 33401, US

(73) Assignee: University of Miami, Miami, FL (US)

(21) Appl. No.: 12/752,802

(22) Filed: Apr. 1, 2010

(30) Foreign Application Priority Data
Oct. 1, 2008 (US) PCT/US08/78455

(51) Int. Cl. A61K 31/7115; C07H 21/02; C07K 14/705; A61P 35/00; A61P 37/06

(52) U.S. Cl. 514/44 A; 536/24.5; 530/358

(57) ABSTRACT

Compositions for countering immune attenuating/suppressive pathways comprise targeting agents or aptamer targeted RNAi-mediated gene silencing (siRNA/shRNA). These compositions have broad applicability in the treatment of many diseases.

### CROSS-REFERENCE TO RELATED APPLICATIONS

The application is a by-pass continuation-in-part, which claims priority of U.S. provisional patent application No. 60/976,603 filed Oct. 1, 2007, and PCT Application No.: PCT/US2008/078445, International filing date Oct. 1, 2008, which are incorporated herein by reference in their entireties.

### FIELD OF THE INVENTION

Embodiments of the invention provide compositions and methods for highly selective targeting of heterologous nucleic acid sequences. The heterologous nucleic acid sequences comprise siRNA's which are targeted to desired cells in vivo and which bind in a sequence dependent manner to their target genes and inhibit expression of undesired nucleic acid sequences in a target cell. The targeting of the siRNA to polynucleotides involved in modulation of an immune response modulates antigen specific immune cell responses.

### BACKGROUND

In 1994, Nilsson and colleagues described an in situ hybridization technique, designated “padlock probes”, which can detect single base mutations yet be seen at the light microscope level (Nilsson, M. et al. “Padlock probes: circularizing oligonucleotides for localized DNA detection”. Science 265, 2085-8 (1994). Padlock probes are large oligonucleotides, whose arms are complementary to, and wrap around the target DNA in an end-to-end orientation, and are then ligated if a perfect match exists between the arms and target. Since both arms are typically about twenty bases each, together they are expected to wrap around a DNA target approximately four times before being locked through ligation (one turn per ˜10 bases). In this way they are inextricably bound to the target (hence “padlock”), permitting highly stringent washing prior to detection, using either the biotin molecules in the non-complementary backbone or through rolling circle amplification.

While existing approaches to target cells based on their genotype is limited, some molecular based approaches have been developed. These include antisense RNA [(Izant, J. G. & Weintraub, H. Science 229, 345-52. (1985); Detrick, B. et al. Invest. Opthalmol. Vis. Sci. 42, 163-9. (2001); Miller, P. S., Cassidy, R. A., Hamma, T. & Kondo, N. S. Pharmacol. Ther. 85, 159-63. (2000)], triplex DNA [(Blume, S. W., Gee, J. E., Shrestha, K. & Miller, D. M. Nucleic Acids Res 20, 1777-84. (1992); Chan, P. P. & Glazer, P. M. J. Mol. Med. 75, 267-82. (1997); Cassidy, R. A., Kondo, N. S. & Miller, P. S. Biochemistry 39, 8683-91. (2000)], ribozymes [(Beaudry, A. A. & Joyce, G. F. Science 257, 635-41. (1992); Joyce, G. F. Science 289, 401-2. (2000)], “suicide” gene therapy [(Shimura, H. et al. Cancer Res. 61, 3640-6. (2001); Black, M. E., Kokoris, M. S. & Sabo, P. Cancer Res. 61, 3022-6. (2000], and inhibitory RNA [(Elbashir, S. M. et al. Nature 411, 494-8 (2001); Brummelkamp, T. R., Bernards, R. & Agami, R. Science 296, 550-3 (2002)].

### SUMMARY

Embodiments of the invention comprises the generation of fusions of aptamer or targeting agents-RNAi's for specifically targeting RNAi to the right cell in vivo. Methods of treatment target lymphocytes wherein these lymphocytes have been suppressed or attenuated. The compositions target various markers, for example, on T lymphocytes, and the RNAi's are specifically delivered to the desired cell population.

Examples of targets on activated T cells are 4-1BB or OX40. Examples of siRNAs targets of suppressive/attenuating pathways are TGFβ receptor, purinergic receptors (for adenosine uptake and conversion to cAMP), CTLA-4, PTEN, Csk, Cb1-b, cytokines, etc.

In a preferred embodiment, a composition for modulating immune cells comprising an aptamer-interference RNA (RNAi) fusion molecule wherein said molecule is targeted to cells and cellular molecules associated with regulation of an immune response.

In another preferred embodiment, the interference RNA comprising at least one of a short interfering RNA (siRNA); a micro, interfering RNA (miRNA); a small, temporal RNA (stRNA); or a short, hairpin RNA (shRNA).

In another preferred embodiment, the immune cells comprise T cells (T lymphocytes), B cells (B lymphocytes), antigen presenting cells, dendritic cells, monocytes, macrophages, myeloid suppressor cells, natural killer (NK) cells, cytotoxic T lymphocytes (CTLs), CTL lines, CTL clones, CTLs from tumor, inflammatory, or other infiltrates and subsets thereof. In some embodiments, the aptamer is specific for T lymphocytes and subsets thereof. For example, the aptamer can target TCR, CD28, CD137, CD137L. Subsets of T lymphocytes are for example, T helper cells, CTLs, Treg.

In another preferred embodiment, the aptamer is specific for CD8⁺T lymphocytes and markers thereof.

In yet another preferred embodiment, the targeting agent or aptamer are specific for T regulatory cells.

In one embodiment, the targeting agent or aptamer are specific for molecules comprising 4-1BB (CD137), OX40, CD3, CD28, HLA-ABC, HLA-DR, T Cell receptor αβ (TCRαβ), T Cell receptor γδ (TCRγδ), T cell receptor ζ (TCRζ), TGFβRII, TNF receptor, Cd11c, CD1-339, B7, mannose receptor, or DEC205, any molecule in Tables 1 to 5, variants, mutants, ligands, alleles and fragments thereof.

In another preferred embodiment, the interference RNA (RNAi) is specific for any one or more polynucleotides comprising TGFβ receptor, TGFβRII, polynucleotides associated with TGFβ signaling, purinergic receptors, CTLA-4, PTEN, Csk, Cb1-b, cytokines, SOCS1, GILT, GILZ, molecules in Tables 1 to 5, A20 or Bax/Bak.

In another preferred embodiment, the RNAi targets TGFβ in activated T lymphocytes.

In another preferred embodiment, the aptamer-RNA interference fusion molecule comprises at least one oligonucleotide as set forth in SEQ ID NOS: 1-6.

In another preferred embodiment, a method of modulating an immune response in patient comprises constructing an aptamer and interference RNA fusion molecule wherein the aptamer is specific for an immune effector cell and the interference RNA is specific for a molecule associated with attenuation or suppression of the immune effector cell; administering the aptamer-interference RNA fusion molecule in a therapeutically effective amount to the patient; and, modulating the immune response.

In another preferred embodiment, the aptamer is specific for an activated CD8⁺T lymphocyte and the interference RNA is specific for TGFβ, variants, mutants and fragments thereof.

In another preferred embodiment, the aptamer-interference RNA comprises at least one of an oligonucleotide as set forth in SEQ ID NOS: 1-6. In preferred embodiments, the aptamer-interference RNA fusion molecule comprises at least one aptamer specific for a desired cell marker for targeting the fusion molecule, and at least one interference RNA molecule specific for a desired polynucleotide.

In yet another embodiment, the aptamer-interference RNA fusion molecule comprises a linker molecule.

In another preferred embodiment, the polynucleotide encoding the aptamer-interference RNA fusion molecule comprises one or more nucleotide substitutions. Preferably, the nucleotide substitutions comprise at least one or combinations thereof, of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants and analogs thereof.

In another preferred embodiment, the linker molecule comprises nucleotide, non-nucleotide, or mixed nucleotide/non-nucleotide linker joining the one or more aptamers to on or more interference RNA molecules.

In a preferred embodiment, the one or more linker molecules comprise about 2 nucleotides length up to about 50 nucleotides in length.

In another preferred embodiment, the non-nucleotide linker comprises abasic nucleotide, polyether, polyamine, polyamide, peptide, carbohydrate, lipid, polyhydrocarbon, or polymeric compounds having 1 or more monomeric units.

In another preferred embodiment, the aptamer-interference RNA molecule comprises at least one aptamer specific for a marker of a target cell and at least one interference RNA molecule specific for a desired polynucleotide of the target cell. Preferably, the at least one aptamer is linked to the at least interference RNA by at least one linker molecule.

In another preferred embodiment, the linker molecule comprises wherein the linker molecule comprises nucleotide, non-nucleotide, or mixed nucleotide/non-nucleotide linker joining the one or more aptamers to on or more interference RNA molecules.

In another preferred embodiment, the one or more linker molecules comprising about 2 nucleotides length up to about 50 nucleotides in length.

In another preferred embodiment, the non-nucleotide linker comprises abasic nucleotide, polyether, polyamine, polyamide, peptide, carbohydrate, lipid, polyhydrocarbon, or polymeric compounds having 1 or more monomeric units. Preferably, the polynucleotide encoding the aptamer-interference RNA fusion molecule comprises one or more nucleotide substitutions. Preferably, the nucleotide substitutions comprise at least one or combinations thereof, of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants and analogs thereof.

In another preferred embodiment, the aptamer is specific for molecules comprising 4-1BB (CD137), OX40, CD3, CD28, or HLA-DR, CD11c, mannose receptor or DEC205variants, mutants, alleles and fragments thereof.

In another preferred embodiment, the interference RNA (RNAi) is specific for polynucleotides comprising TGFβ receptor, polynucleotides associated with TGFβ signaling, purinergic receptors, CTLA-4, PTEN, Csk, Cb1-b, cytokines, SOCS1, GILT, GILZ, A20 or Bax/Bak.

In yet another embodiment, the aptamer is specific for 4-1BB (CD137), OX40, CD3, CD28, HLA-ABC, HLA-DR, T Cell receptor αβ (TCRαβ), T Cell receptor γδ (TCRγδ), T cell receptor ζ (TCRζ), TNF receptor, Cd11c, CD1-339, B7, mannose receptor, or DEC205, variants, mutants, ligands, alleles and fragments thereof.

In another embodiment, the interference RNA comprising at least one of a short interfering RNA (siRNA); a micro, interfering RNA (miRNA); a small, temporal RNA (stRNA); or a short, hairpin RNA (shRNA).

Other aspects are described infra.

### BRIEF DESCRIPTION OF THE DRAWINGS

FIG. 1 is a schematic representation showing isolation of aptamers using “systematic evolution of ligands by exponential enrichment” (SELEX). The starting point for the in vitro selection process is a combinatorial modified RNA. To isolate high affinity nucleic acid ligands to a given target protein the starting library of aptamer is incubated with the protein of interest. Nucleic acid molecules that bind to a specific protein are then partitioned from other sequences in the library, the bound sequences are removed from the protein and amplified by reverse transcription and PCR to generate a library enriched in sequences that bind to the target protein. This library is then transcribed in vitro to generate molecules for use in the nest round of selection. After several rounds the selected ligands are sequenced and evaluated for their affinity for the targeted protein.

FIGS. 2A-2B is a schematic representation showing some embodiments of a design of aptamer-siRNA chimeras. FIG. 2A is a schematic diagram of a dual-function immunomodulatory oligonucleotide. An oligonucleotide aptamer which binds to 4-1BB is joined to a CTLA-4 siRNA and inhibition of CTLA-4 expression. FIG. 2B is a schematic representation showing an aptamer dimer with siRNA in either of two positions. The dimeric forms of aptamer will not only bind to 4-1BB but will also transmit a costimulatory signal.

FIG. 3 is a scan of a photograph showing the downregulation of CTLA-4 in polyclonal activated CD8⁺ cells incubated with a monomeric 4-1BB aptamer-CTLA-4 siRNA chimera. Cells were also incubated with control chimeras containing a mutant non-binding 4-1BB or non aptamer chimera. The mRNA content was determined by RT-PCR.

FIGS. 4A, 4B show enhanced activation of CD8⁺T cells incubated monomeric aptamer—CTLA-4 siRNA chimeras. FIG. 4A: Proliferation measured using the CFSE dilution assay FIG. 4B: IL-2 secretion determined by ELISA.

FIGS. 5A-5D show the functional characterization of a dual function 4-1 BB aptamer CTLA-4 siRNA chimeric ODN. FIG. 5A: A second 4-1 BB aptamer was conjugated to the 5′ end of the 4-1 BB aptamer-siRNA chimeric molecule. FIG. 5B shows enhanced IL-2 secretion when 4-1 BB aptamer dimer is conjugated to a CTLA-4 siRNA compared to control siRNA. FIG. 5C: 4-1 BB co-stimulation was determined by measuring proliferation when cells are incubated with either 4-1 BB aptamer dimer—control siRNA or anti-4-1 BB antibody (3H3). FIG. 5D shows the additive effect of 4-1 BB co-stimulation and CTLA-4 blockade mediated by 4-1 BB aptamer dimer—CTLA-4 siRNA chimeras. CD3 stimulated CD8⁺T cells were incubated with either 4-1 BB aptamer-dimer—control siRNA or 4-1 BB aptamer dimer—CTLA-4 siRNA and proliferation was measured as described except that incubation was extended two more days to monitor for cells that underwent more extensive proliferation. αCD3 panel—no aptamers—siRNA chimeras. IgG panel—αCD3 antibody was replaced with isotype matched antibody and 4-1BB aptamer dimer CTLA-4 siRNA.

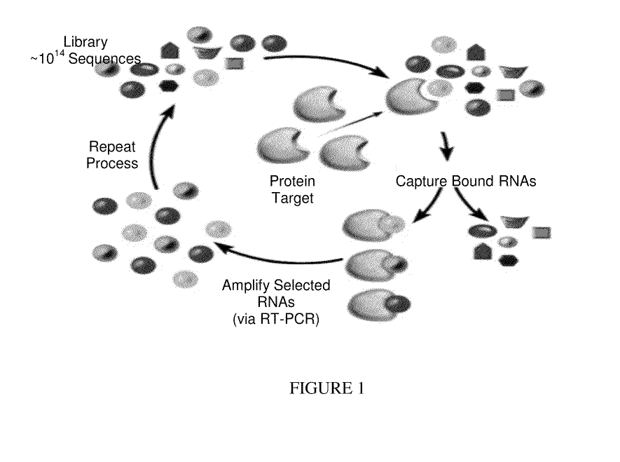

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the terms “oligonucleotide,” “siRNA,” “siRNA oligonucleotide,” and “siRNA's” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoögsteen or reverse Hoögsteen types of base pairing, or the like.

The oligonucleotide may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register,” that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139 (1999); Freier S., M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000),); 2′-O, 3′-C-linked [3.2.0] bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

As used herein, the term “gene” means the gene and all currently known variants thereof and any further variants which may be elucidated.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the term “mRNA” means the presently known mRNA transcript(s) of a targeted gene, and any further transcripts which may be elucidated.

By “desired RNA” molecule is meant any foreign RNA molecule which is useful from a therapeutic, diagnostic, or other viewpoint. Such molecules include antisense RNA molecules, decoy RNA molecules, enzymatic RNA, therapeutic editing RNA and agonist and antagonist RNA.

By “antisense RNA” is meant a non-enzymatic RNA molecule that binds to another RNA (target RNA) by means of RNA-RNA interactions and alters the activity of the target RNA (Eguchi et al., 1991 Annu. Rev. Biochem. 60, 631-652).

RNA interference “RNAi” is mediated by double stranded RNA (dsRNA) molecules that have sequence-specific homology to their “target” nucleic acid sequences (Caplen, N. J., et al., Proc. Natl. Acad. Sci. USA 98:9742-9747 (2001)). In certain embodiments of the present invention, the mediators of RNA-dependent gene silencing are 21-25 nucleotide “small interfering” RNA duplexes (siRNAs). The siRNAs are derived from the processing of dsRNA by an RNase enzyme known as Dicer (Bernstein, E., et al., Nature 409:363-366 (2001)). siRNA duplex products are recruited into a multi-protein siRNA complex termed RISC (RNA Induced Silencing Complex). Without wishing to be bound by any particular theory, a RISC is then believed to be guided to a target nucleic acid (suitably mRNA), where the siRNA duplex interacts in a sequence-specific way to mediate cleavage in a catalytic fashion (Bernstein, E., et al., Nature 409:363-366 (2001); Boutla, A., et al., Curr. Biol. 11:1776-1780 (2001)). Small interfering RNAs that can be used in accordance with the present invention can be synthesized and used according to procedures that are well known in the art and that will be familiar to the ordinarily skilled artisan. Small interfering RNAs for use in the methods of the present invention suitably comprise between about 0 to about 50 nucleotides (nt). In examples of nonlimiting embodiments, siRNAs can comprise about 5 to about 40 nt, about 5 to about 30 nt, about 10 to about 30 nt, about 15 to about 25 nt, or about 20-25 nucleotides.

Selection of appropriate RNAi is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

By “enzymatic RNA” is meant an RNA molecule with enzymatic activity (Cech, 1988 J. American. Med. Assoc. 260, 3030-3035). Enzymatic nucleic acids (ribozymes) act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through base-pairing, and once bound to the correct site, acts enzymatically to cut the target RNA.

By “decoy RNA” is meant an RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is meant to be a specific example. Those in the art will recognize that this is but one example, and other embodiments can be readily generated using techniques generally known in the art.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 80% or 90%, preferably 95%, most preferably 100%, complementarity to a defined sequence. Preferably, alleles or variants thereof can be identified. A BLAST program also can be employed to assess such sequence identity.

The term “complementary sequence” as it refers to a polynucleotide sequence, relates to the base sequence in another nucleic acid molecule by the base-pairing rules. More particularly, the term or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99% to about 100%. Complementary polynucleotide sequences can be identified by a variety of approaches including use of well-known computer algorithms and software, for example the BLAST program.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions, e.g., as described below, is a convenient measure of duplex and/or triplex stability. Preferably, oligonucleotides of the invention are selected that have melting temperatures of at least 45° C. when measured in 100 mM NaCl, 0.1 mM EDTA and 10 mM phosphate buffer aqueous solution, pH 7.0 at a strand concentration of both the oligonucleotide and the target nucleic acid of 1.5 μM. Thus, when used under physiological conditions, duplex or triplex formation will be substantially favored over the state in which the antigen and its target are dissociated. It is understood that a stable duplex or triplex may in some embodiments include mismatches between base pairs and/or among base triplets in the case of triplexes. Preferably, modified oligonucleotides, e.g. comprising LNA units, of the invention form perfectly matched duplexes and/or triplexes with their target nucleic acids.

As used herein, the term “Thermal Melting Point (Tm)” refers to the temperature, under defined ionic strength, pH, and nucleic acid concentration, at which 50% of the oligonucleotides complementary to the target sequence hybridize to the target sequence at equilibrium. As the target sequences are generally present in excess, at Tm, 50% of the oligonucleotides are occupied at equilibrium). Typically, stringent conditions will be those in which the salt concentration is at least about 0.01 to 1.0 M Na ion concentration (or other salts) at pH 7.0 to 8.3 and the temperature is at least about 30° C. for short oligonucleotides (e.g., 10 to 50 nucleotide). Stringent conditions may also be achieved with the addition of destabilizing agents such as formamide.

The term “stringent conditions” refers to conditions under which an oligonucleotide will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

The term “target nucleic acid” refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA) whose expression level it is desired to detect. The difference in usage will be apparent from context.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

As used herein, a “pharmaceutically acceptable” component/carrier etc is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

The term “targeting agent” refers to a molecule which specifically binds to another molecule. For example, an antibody or fragments thereof, aptamers, RGD peptides, integrins, receptors or ligands, or any other molecule that can specifically bind to a target molecule.

The term “specifically binds” to a target molecule, such as for example, an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. For example, an antibody “specifically binds” or “preferentially binds” to a target if it binds with greater affinity, avidity, more readily, and/or with greater duration than it binds to other substances. It is also understood by reading this definition that; for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3^{rd} ed. Cold Spring Harbor Laboratory Press: Cold Spring Harbor, N.Y.; Sambrook et al., (1989) Molecular Cloning: A Laboratory Manual. 2^{nd} ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4^{th} ed. Wiley-Liss; among others. The Current Protocols listed above are updated several times every year.

“Target molecule” includes any macromolecule, including protein, carbohydrate, enzyme, polysaccharide, glycoprotein, receptor, antigen, antibody, growth factor; or it may be any small organic molecule including a hormone, substrate, metabolite, cofactor, inhibitor, drug, dye, nutrient, pesticide, peptide; or it may be an inorganic molecule including a metal, metal ion, metal oxide, and metal complex; it may also be an entire organism including a bacterium, virus, and single-cell eukaryote such as a protozoon.

### Compositions

Delivery of RNAi in vivo could overcome attenuation/suppression and result in more potent immunity. However, non-targeted delivery of RNAi in vivo was not, heretofore, clinically practical because of cost consideration and anticipated toxicity. Embodiments of the present invention comprise targeting RNAi to the appropriate cells, antigen-activated T cells in this instance, to solve the problems with modulating immune effector cell response. Use of antibodies for generally targeting RNAi in vivo has not be efficacious. Antibodies are cell based products, and pose significant cost, manufacturing, and regulatory challenges. However, many targeting agents, RGD based peptides, integrins, can be used. Antibodies can also be used, although they are not as desirable.

In preferred embodiments, aptamers specifically target, for example, siRNA, to a desired nucleic acid target. Aptamers are oligonucleotide-based ligands that exhibit specificity and avidity comparable or superior to antibodies. However, unlike antibodies, aptamers are synthesized chemically in cell free system, and offer a more straightforward and cost effective manufacturing process and a vastly simpler regulatory approval process for clinical use.

In a preferred embodiment, the compositions of the present invention are targeted to the cells involved in modulation of the immune system, such as, for example, immune effector cells, cells involved in the regulation of the immune system, e.g. T regulatory cells (Treg), MSC, antigen presenting cells and the like. Examples of antigen presenting cells include, dendritic cells, b cells, momocytes/macrophages.

Immune System: Immune systems are classified into two general systems, the “innate” or “primary” immune system and the “acquired/adaptive” or “secondary” immune system. It is thought that the innate immune system initially keeps the infection under control, allowing time for the adaptive immune system to develop an appropriate response. Studies have suggested that the various components of the innate immune system trigger and augment the components of the adaptive immune system, including antigen-specific B and T lymphocytes (Kos, Immunol. Res. 1998, 17:303; Romagnani, Immunol. Today. 1992, 13: 379; Banchereau and Steinman, Nature. 1988, 392:245).

A “primary immune response” refers to an innate immune response that is not affected by prior contact with the antigen. The main protective mechanisms of primary immunity are the skin (protects against attachment of potential environmental invaders), mucous (traps bacteria and other foreign material), gastric acid (destroys swallowed invaders), antimicrobial substances such as interferon (IFN) (inhibits viral replication) and complement proteins (promotes bacterial destruction), fever (intensifies action of interferons, inhibits microbial growth, and enhances tissue repair), natural killer (NK) cells (destroy microbes and certain tumor cells, and attack certain virus infected cells), and the inflammatory response (mobilizes leukocytes such as macrophages and dendritic cells to phagocytose invaders).

Some cells of the innate immune system, including macrophages and dendritic cells (DC), function as part of the adaptive immune system as well by taking up foreign antigens through pattern recognition receptors, combining peptide fragments of these antigens with major histocompatibility complex (MHC) class I and class II molecules, and stimulating naive CD8⁺ and CD4⁺T cells respectively (Banchereau and Steinman, supra; Holmskov et al., Immunol. Today. 1994, 15:67; Ulevitch and Tobias Annu. Rev. Immunol. 1995, 13:437). Professional antigen-presenting cells (APCs) communicate with these T cells, leading to the differentiation of naive CD4⁺T cells into T-helper 1 (Th1) or T-helper 2 (Th2) lymphocytes that mediate cellular and humoral immunity, respectively (Trinchieri Annu. Rev. Immunol. 1995, 13:251; Howard and O'Gana, Immunol. Today. 1992, 13:198; Abbas et al., Nature. 1996, 383:787; Okamura et al., Adv. Immunol. 1998, 70:281; Mosmann and Sad, Immunol. Today. 1996, 17:138; O'Garra Immunity. 1998, 8:275).

A “secondary immune response” or “adaptive immune response” may be active or passive, and may be humoral (antibody based) or cellular that is established during the life of an animal, is specific for an inducing antigen, and is marked by an enhanced immune response on repeated encounters with said antigen. A key feature of the T lymphocytes of the adaptive immune system is their ability to detect minute concentrations of pathogen-derived peptides presented by MHC molecules on the cell surface. Upon activation, naïve CD4 T cells differentiate into one of at least two cell types, Th1 cells and Th2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response, and reciprocally, activation of the Th2 type cytokine response can suppress the Th1 type response.

In adaptive immunity, adaptive T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

The phrase “T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response.

“Cells of the immune system” or “immune cells”, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhan's cells, stem cells, dendritic cells, peripheral blood mononuclear cells, tumor-infiltrating (TIL) cells, gene modified immune cells including hybridomas, drug modified immune cells, antigen presenting cells and derivatives, precursors or progenitors of the above cell types.

“Immune effector cells” refers to cells, and subsets thereof, e.g. Treg, Th1, Th2, capable of binding an antigen and which mediate an immune response selective for the antigen. These cells include, but are not limited to, T cells (T lymphocytes), B cells (B lymphocytes), antigen presenting cells, such as for example dendritic cells, monocytes, macrophages; myeloid suppressor cells, natural killer (NK) cells and cytotoxic T lymphocytes (CTLs), for example CTL lines, CTL clones, and CTLs from tumor, inflammatory, or other infiltrates.

A “T regulatory cell” or “Treg cell” or “Tr cell” refers to a cell that can inhibit a T cell response. Treg cells express the transcription factor Foxp3, which is not upregulated upon T cell activation and discriminates Tregs from activated effector cells. Tregs are identified by the cell surface markers CD25, CD45RB, CTLA4, and GITR. Treg development is induced by MSC activity. Several Treg subsets have been identified that have the ability to inhibit autoimmune and chronic inflammatory responses and to maintain immune tolerance in tumor-bearing hosts. These subsets include interleukin 10-(IL-10-) secreting T regulatory type 1 (Tr1) cells, transforming growth factor-β-(TGF-β-) secreting T helper type 3 (Th3) cells, and “natural” CD4⁺/CD25⁺ Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

The term “myeloid suppressor cell (MSC)” refers to a cell that is of hematopoietic lineage and expresses Gr-1 and CD11b; MSCs are also referred to as immature myeloid cells and were recently renamed to myeloid-derived suppressor cells (MDSCs). MSCs may also express CD115 and/or F4/80 (see Li et al., Cancer Res. 2004, 64:1130-1139). MSCs may also express CD31, c-kit, vascular endothelial growth factor (VEGF)-receptor, or CD40 (Bronte et al., Blood. 2000, 96:3838-3846). MSCs may further differentiate into several cell types, including macrophages, neutrophils, dendritic cells, Langerhan's cells, monocytes or granulocytes. MSCs may be found naturally in normal adult bone marrow of human and animals or in sites of normal hematopoiesis, such as the spleen in newborn mice. Upon distress due to graft-versus-host disease (GVHD), cyclophosphamide injection, or γ-irradiation, for example, MSCs may be found in the adult spleen. MSCs can suppress the immunological response of T cells, induce T regulatory cells, and produce T cell tolerance. Morphologically, MSCs usually have large nuclei and a high nucleus-to-cytoplasm ratio. MSCs can secrete TFG-β and IL-10 and produce nitric oxide (NO) in the presence of IFN-γ or activated T cells. MSCs may form dendriform cells; however, MSCs are distinct from dendritic cells (DCs) in that DCs are smaller and express CD11c; MSCs do not express CD11c. T cell inactivation by MSCs in vitro can be mediated through several mechanisms: IFN-γ-dependent nitric oxide production (Kusmartsev et al. J Immunol. 2000, 165: 779-785); Th2-mediated-IL-4/IL-13-dependent arginase 1 synthesis (Bronte et al. J Immunol. 2003, 170: 270-278); loss of CD3ξ signaling in T cells (Rodriguez et al. J Immunol. 2003, 171: 1232-1239); and suppression of the T cell response through reactive oxygen species (Bronte et al. J Immunol. 2003, 170: 270-278; Bronte et al. Trends Immunol. 2003, 24: 302-306; Kusmartsev et al. J Immunol. 2004, 172: 989-999; Schmielau and Finn, Cancer Res. 2001, 61: 4756-4760).

Potentiating tumor immunity using aptamer-mediated targeting of immunomodulatory siRNAs: Limited specificity of drugs and the need to reach all, or the vast majority, of the tumor cells disseminated throughout the body are the two major challenges in developing effective treatments for cancer. Mechanistic studies of tumorigenesis at the molecular and cellular levels have stimulated new paradigms of increasingly sophisticated large-scale drug screening programs. A complementary, and a more general, approach to increase the specificity of otherwise poorly specific drugs is to target the drug to the right cells in the body, the cancer cells or cancer stem cells. Antibodies have been the choice as targeting ligands, yet the development of antibody-targeted chemotherapy, “immunotoxins”, has been slow. Several reasons account for this, including poor penetration into the solid tumor, a vascular leak syndrome caused by the high concentration of immunotoxin, and immunogenicity of the antibody. Foremost, since antibodies are cell-based products, their use in clinical setting is posing significant cost, manufacturing, and regulatory challenges. Hence clinical-grade antibodies are almost exclusively developed and provided by companies on a selective basis and under strict contractual agreement. Thus, despite promising observations from murine preclinical tumor models, the use of antibody-based reagents in human patients is significantly limited.

In preferred embodiments, modulation of immune cells and subsequent responses comprises a method of treating a patient with cancer wherein an siRNA is specifically targeted and delivered to a cell in order to modulate the functions of that cells, for example, proliferation of a lymphocyte wherein that lymphocyte had been previously suppressed or attenuated. The cells of the immune system are regulated by both cellular and soluble factors, e.g. cytokines, growth factors and the like. Thus, in some embodiments, the compositions of the invention are targeted to polynucleotides encoding products responsible for down regulating or suppressing a cell involved in an immune response. The cell can be any type of one or more immune cells. In some preferred embodiments, the immune cell is a lymphocyte. These reagents or compositions involved or associated with modulating immunity, such as costimulation (i.e., CTLA-4, 4-1BB, PD-1, etc.) or TGFβ-mediated suppression, serve as important adjunct to, or replace altogether, new and powerful, often complex, vaccination protocols currently under development.

The compositions also comprise one or more aptamers or targeting agents specific for at least one molecule. Thus, the molecules can be poly-specific. For example, an aptamer may be specific for a desired molecule and a second aptamer which is also part of the aptamer-interference RNA molecule can be specific for another molecule.

Negative regulatory pathway, and not lack of inherent tumor immunogenicity (i.e., the ability of the unmanipulated tumors to stimulate protective immunity), play an important role in preventing the immune-mediated control of tumor progression. The therapeutic implication is that countering immune-attenuating/suppressive regulatory circuits contributes to successful immune control of cancer and is as, if not more, important than developing potent vaccination protocols.

In a preferred embodiment, a composition comprising a targeting agent and a gene silencing agent down-regulate or abrogate immune attenuating/suppressive pathways. In a preferred embodiment, the gene silencing agent is an RNAi (siRNA/shRNA).

In a preferred embodiment, the gene silencing agent (the RNAi) is targeted to the appropriate immune cells in vivo using nuclease-resistant oligonucleotide-based aptamers. Targeting of polynucleotides involved in the modulation of an immune response includes, without limitation, any one or more components of a pathway that suppresses an immune response. For example, any one or more components of the TGF-β mediated pathway which leads to the suppression of an immune response.

An important distinction between drugs which target the cancer cell directly and immunomodulatory agents is that in order for the cancer drug to be effective it has to reach and eliminate the vast majority of tumor cells disseminated throughout the body. By contrast, the immunomodulatory agents will be effective if they reach a fraction of the immune cells because the ensuing antitumor immune response is systemic. Thus whether targeting or not, immune-potentiating drugs do not have to reach all the target cells in vivo. This has important implications, reduced cost and less toxicity, because in all likelihood the amount of immunomodulatory agent that need to be injected will be significantly less than that of agents targeting the tumor cell directly.

In a preferred embodiment, the aptamer-siRNA composition is targeted to activated T cells. The aptamer is specific for an activated T cell marker so as to specifically deliver the siRNA to the intended target, in this embodiment, polynucleotides involved in the TGFβ signaling pathway. Progressing tumors often secrete TGFβ and TGFβ signaling in tumor infiltrating CD8⁺T cells attenuates their function. In murine tumor models, TGFβ signaling in tumor specific CD8⁺T cells is the primarily mechanism responsible for tumor outgrowth (because interfering with TGFβ signaling using dominant-negative TGFβRII-expressing CD8⁺T cells can abrogate the growth of poorly immunogenic tumors even in the absence of vaccination). Inhibition TGFβ signaling in vaccine-induced activated T cells, but not other cells in the body most of which express TGFβ receptor, represents a powerful means of potentiating tumor immunity. For example, as described in the examples which follow, an aptamer was developed, which binds to and inhibits the function of the negative costimulatory receptor CTLA-4. Another target for which an aptamer was developed was 4-1BB (CD137). In one embodiment, the aptamer which targets TGFβ siRNA to activated T cells is the 4-1BB aptamer. 4-1BB is upregulated on antigen-activated T cells.

In another preferred embodiment, the aptamer-RNAi compositions are administered to a patient either alone or part of another therapy. For example, in the case of treating a patient with cancer, the aptamer-RNAi composition can be administered with, prior to or after, treatments such as chemotherapy, surgery, radiation and the like.

In a preferred embodiment, RNAi comprising siRNA or shRNA, inhibit TGFβRII or other components of the TGFβ signaling pathway in activated T cells. In a preferred embodiment, the RNAi composition is specifically targeted to a desired cell, for example, activated T cell. Non-targeted delivery of siRNA/shRNA in vivo would otherwise require large quantities of reagent which will be cost-prohibitive and likely to be accompanied by rate limiting toxicities. On the other hand, targeting siRNA to the relevant cells, in this embodiment, activated T cells, drastically reduced the amount of siRNA needed to infuse in the patient and the potential for adverse effects.

In another preferred embodiment, the compositions comprising aptamer-siRNA are targeted to pathways which are involved in mediating a shift between Th1 and Th2 immune responses. For example, the cytotoxic T cells may be shut down or suppressed. CD8+T cells are important in combating tumors and cells infected with foreign agents such as for example, viruses. Both tumors and viruses have been shown to manipulate the immune response in many ways, e.g HIV. Thus, the aptamer-RNAi compositions can be targeted to those cells and regulatory pathways that suppress the CD8⁺T cell response. For example, regulation of pathways of CD IFN-γ, GM-CSF, TNF-α, CD40 ligand, Fas ligand, IL-3, TNF-β, and IL-2. Cells targeted include one or more types of Treg cells, antigen presenting cells and the like.

Thus in a preferred embodiment, aptamer-RNAi compositions for modulating, for example, tumor immunity, are employed for silencing TGFβ signaling in activated T cells. Other applications include, but not limited to:

Inhibiting attenuation of T cell receptor (TCR) signaling mediated by CTLA-4, PTEN, Cb1-b, Csk, cAMP pathways, etc, involved in the immune response of tumor immunity, including TGFβ pathway.

Inhibition of dendritic cell-intrinsic attenuation pathways such as pathways mediated by SOCS1, GILT, Bax/Bak, etc., using aptamers directed for example to CD11c, mannose receptor or DEC205.

Inhibition of Treg function by inactivating Foxp3 using aptamer targeted RNAi (for example aptamers corresponding to 4-1BB or OX-40 which are also expressed on Treg).

Controlling GVHD in the setting of allotransplantation of hematologic malignancies by eliminating activated T cells using aptamer-guided RNAi corresponding to survival genes such as Bcl-2 and others. In some embodiments, the aptamer-RNAi composition is directed to cells and pathways involved in transplantation rejection and autoimmune responses.

In another preferred embodiment, the aptamer-RNAi compositions target cells and pathways involved in rendering the immune system tolerant to a particular antigen or antigens. “Tolerance” refers to the anergy (non-responsiveness) of immune cells, e.g. T cells, when presented with an antigen. T cell tolerance prevents a T cell response even in the presence of an antigen that existing memory T cells recognize.

In another preferred embodiment, the siRNA can be used in treating diseases wherein immune cells are involved in the disease, such as, autoimmune diseases; hypersensitivity to allergens; organ rejection; inflammation; and the like. Generally, these are conditions in which the immune system of an individual (e.g., activated T cells) attacks the individual's own tissues and cells, or implanted tissues, cells, or molecules (as in a graft or transplant). Exemplary autoimmune diseases that can be treated with the methods of the instant disclosure include type I diabetes, multiple sclerosis, thyroiditis (such as Hashimoto's thyroiditis and Ord's thyroiditis), Grave's disease, systemic lupus erythematosus, scleroderma, psoriasis, arthritis, rheumatoid arthritis, alopecia greata, ankylosing spondylitis, autoimmune hemolytic anemia, autoimmune hepatitis, Behçet's disease, Crohn's disease, dermatomyositis, glomerulonephritis, Guillain-Barré syndrome, inflammatory bowel disease, lupus nephritis, myasthenia gravis, myocarditis, pemphigus/pemphigoid, pernicious anemia, polyarteritis nodosa, polymyositis, primary biliary cirrhosis, rheumatic fever, sarcoidosis, Sjögren's syndrome, ulcerative colitis, uveitis, vitiligo, and Wegener's granulomatosis. Exemplary alloimmune responses that can be treated with the methods of the instant disclosure include graft-versus host disease, graft versus leukemia and transplant rejection. Examples of inflammation associated with conditions such as: adult respiratory distress syndrome (ARDS) or multiple organ injury syndromes secondary to septicemia or trauma; reperfusion injury of myocardial or other tissues; acute glomerulonephritis; reactive arthritis; dermatoses with acute inflammatory components; acute purulent meningitis or other central nervous system inflammatory disorders; thermal injury; hemodialysis; leukapheresis; ulcerative colitis; Crohn's disease; necrotizing enterocolitis; granulocyte transfusion associated syndromes; and cytokine-induced toxicity.

The methods of the invention can be used to screen for siRNA polynucleotides that inhibit the functional expression of one or more genes that modulate immune related molecule expression. For example, the CD-18 family of molecules is important in cellular adhesion. Through the process of adhesion, lymphocytes are capable of continually monitoring an animal for the presence of foreign antigens. Although these processes are normally desirable, they are also the cause of organ transplant rejection, tissue graft rejection and many autoimmune diseases. Hence, siRNA's capable of attenuating or inhibiting cellular adhesion would be highly desirable in recipients of organ transplants (for example, kidney transplants), tissue grafts, or for autoimmune patients.

In another preferred embodiment, siRNA oligonucleotides inhibit the expression of MHC molecules involved in organ transplantation or tissue grafting. For example, Class I and Class II molecules of the donor. siRNA inhibit the expression of these molecules thereby ameliorating an allograft reaction. Immune cells may be treated prior to the organ or tissue transplantation, administered at time of transplantation and/or any time thereafter, at times as may be determined by an attending physician. siRNAs can be administered with or without immunosuppressive drug therapy.

The term “transplant” includes any cell, organ, organ system or tissue which can elicit an immune response in a recipient subject mammal. In general, therefore, a transplant includes an allograft or a xenograft cell, organ, organ system or tissue. An allograft refers to a graft (cell, organ, organ system or tissue) obtained from a member of the same species as the recipient. A xenograft refers to a graft (cell, organ, organ system or tissue) obtained from a member of a different species as the recipient. The term “immune rejection,” as used herein, is intended to refer to immune responses involved in transplant rejection, as well as to the concomitant physiological result of such immune responses, such as for example, interstitial fibrosis, chronic graft artheriosclerosis, or vasculitis. The term “immune rejection,” as used herein, is also intended to refer to immune responses involved in autoimmune disorders, and the concomitant physiological result of such immune responses, including T cell-dependent infiltration and direct tissue injury; T cell-dependent recruitment and activation of macrophages and other effector cells; and T cell-dependent B cell responses leading to autoantibody production.

Feasibility, generality, and potential of using aptamer targeted siRNA/gene silencing to modulate antitumor immunity: The use of aptamer-siRNA to manipulate tumor immunity is directed to tumor-orchestrated immune attenuating/suppressive pathways playing a major role in preventing immune mediated control of tumor progression. Use of aptamers to target gene silencing to the appropriate cells in vivo provides a drug/reagent that can be chemically synthesized in cell-free systems which significantly enhances the clinical applicability of this targeting approach (compared to antibody-based targeting), drastically reducing the amount of siRNA reagent needed for treatment and consequently the cost-effectiveness and toxicity of the treatment. Furthermore, a key advantage of immune modulating drugs, whether targeted or not, is that only a fraction of the target cells need to be accessed in vivo for the approach to be successful.

Lastly, aptamer-siRNA technology can be used to enhance the immunogenicity and antigenicity of disseminated tumor by targeting, in this instance the tumor cells (need not be at high efficiency), with siRNAs to promote calreticulin (CRT) driven “immunogenic death” or expression of novel antigens thru inhibition of nonsense mediated decay (NMD).

Generation of Interference RNA: Detailed methods of producing the RNAi's are described in the examples section which follows. The RNAi's of the invention can also be obtained using a number of techniques known to those of skill in the art. For example, the siRNA can be chemically synthesized or recombinantly produced using methods known in the art, such as the Drosophila in vitro system described in U.S. published application 2002/0086356 of Tuschl et al., the entire disclosure of which is herein incorporated by reference.

Preferably, the RNAi's of the invention are chemically synthesized using appropriately protected ribonucleotide phosphoramidites and a conventional DNA/RNA synthesizer. The RNAi can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, RNAi can also be expressed from recombinant circular or linear DNA plasmids using any suitable promoter. Suitable promoters for expressing RNAi of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the RNAi in a particular tissue or in a particular intracellular environment. RNAi's of the invention can be expressed from a recombinant plasmid either as two separate, complementary RNA molecules, or as a single RNA molecule with two complementary regions.

Selection of plasmids suitable for expressing RNAi of the invention, methods for inserting nucleic acid sequences for expressing the RNAi into the plasmid, and methods of delivering the recombinant plasmid to the cells of interest are within the skill in the art. See, for example Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16:948-958; Lee N S et al, (2002), Nat. Biotechnol. 20: 500-505; and Paul C P et al. (2002), Nat. Biotechnol. 20: 505-508, the entire disclosures of which are herein incorporated by reference.

As used herein, “in operable connection with a polyT termination sequence” means that the nucleic acid sequences encoding the sense or antisense strands are immediately adjacent to the polyT termination signal in the 5′ direction. During transcription of the sense or antisense sequences from the plasmid, the polyT termination signals act to terminate transcription.

As used herein, “under the control” of a promoter means that the nucleic acid sequences encoding the sense or antisense strands are located 3′ of the promoter, so that the promoter can initiate transcription of the sense or antisense coding sequences.

Any viral vector capable of accepting the coding sequences for the siRNA molecule(s) to be expressed can be used, for example vectors derived from adenovirus (AV); adeno-associated virus (AAV); retroviruses (e.g., lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, methods for inserting nucleic acid sequences for expressing the RNAi into the vector, and methods of delivering the viral vector to the cells of interest are within the skill in the art. See, for example, Dornburg R (1995), Gene Therap. 2: 301-310; Eglitis M A (1998), Biotechniques 6: 608-614; Miller A D (1990), Hum Gene Therap. 1: 5-14; and Anderson W F (1998), Nature 392: 25-30, the entire disclosures of which are herein incorporated by reference.

A suitable AV vector for expressing the RNAi's of the invention, a method for constructing the recombinant AV vector, and a method for delivering the vector into target cells, are described in Xia H et al. (2002), Nat. Biotech. 20: 1006-1010. Suitable AAV vectors for expressing the RNAi's of the invention, methods for constructing the recombinant AAV vector, and methods for delivering the vectors into target cells are described in Samulski R et al. (1987), J. Virol. 61: 3096-3101; Fisher K J et al. (1996), J. Virol., 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosure of which are herein incorporated by reference.

The ability of an RNAi containing a given target sequence to cause RNAi-mediated degradation of the target mRNA can be evaluated using standard techniques for measuring the levels of RNA or protein in cells. For example, RNA of the invention can be delivered to cultured cells, and the levels of target mRNA can be measured by Northern blot or dot blotting techniques, or by quantitative RT-PCR. RNAi-mediated degradation of target mRNA by an siRNA containing a given target sequence can also be evaluated with animal models, such as mouse models. RNAi-mediated degradation of the target mRNA can be detected by measuring levels of the target mRNA or protein in the cells of a subject, using standard techniques for isolating and quantifying mRNA or protein as described above.

In a preferred embodiment, siRNA molecules target overlapping regions of a desired sense/antisense locus, thereby modulating both the sense and antisense transcripts. In another preferred embodiment, a composition comprises siRNA molecules, of either one or more, and/or, combinations of siRNAs, siRNAs that overlap a desired target locus, and/or target both sense and antisense (overlapping or otherwise). These molecules can be directed to any target that is desired for potential therapy of any disease or abnormality. Theoretically there is no limit as to which molecule is to be targeted. Furthermore, the technologies taught herein allow for tailoring therapies to each individual.

In preferred embodiments, the oligonucleotides can be tailored to individual therapy, for example, these oligonucleotides can be sequence specific for allelic variants in individuals, the up-regulation or inhibition of a target can be manipulated in varying degrees, such as for example, 10%, 20%, 40%, 100% expression relative to the control. That is, in some patients it may be effective to increase or decrease target gene expression by 10% versus 80% in another patient.

Up-regulation or inhibition of gene expression may be quantified by measuring either the endogenous target RNA or the protein produced by translation of the target RNA. Techniques for quantifying RNA and proteins are well known to one of ordinary skill in the art. In certain preferred embodiments, gene expression is inhibited by at least 10%, preferably by at least 33%, more preferably by at least 50%, and yet more preferably by at least 80%. In particularly preferred embodiments, of the invention gene expression is inhibited by at least 90%, more preferably by at least 95%, or by at least 99% up to 100% within cells in the organism. In certain preferred embodiments, gene expression is up-regulated by at least 10%, preferably by at least 33%, more preferably by at least 50%, and yet more preferably by at least 80%. In particularly preferred embodiments, of the invention gene expression is up-regulated by at least 90%, more preferably by at least 95%, or by at least 99% up to 100% within cells in the organism.

Selection of appropriate RNAi is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of RNAi that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

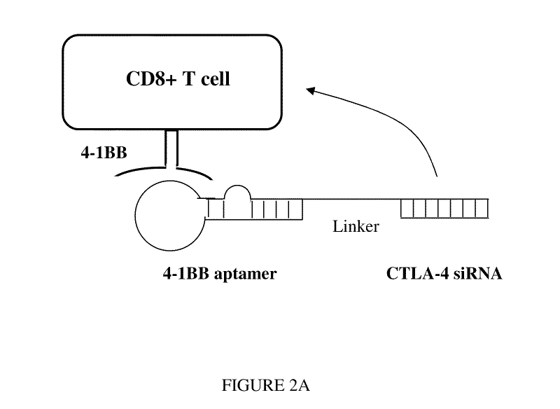

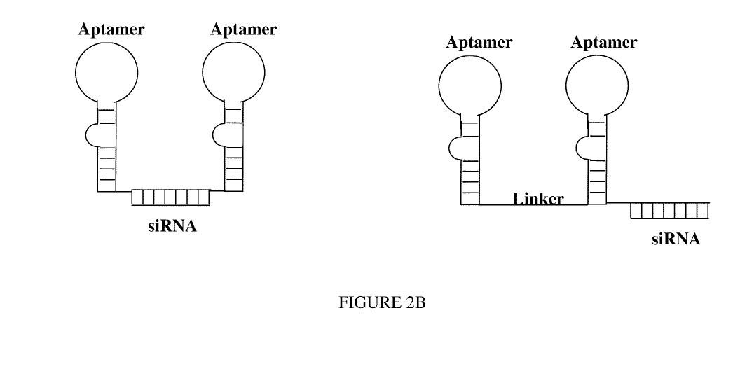

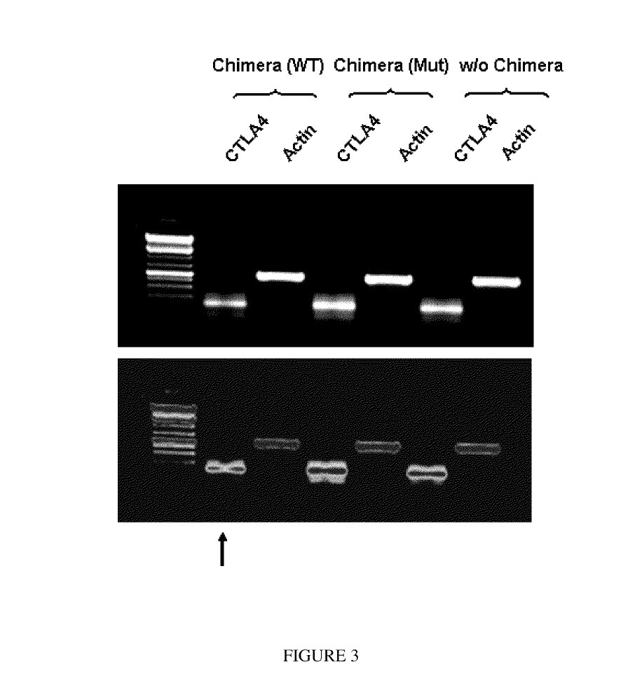

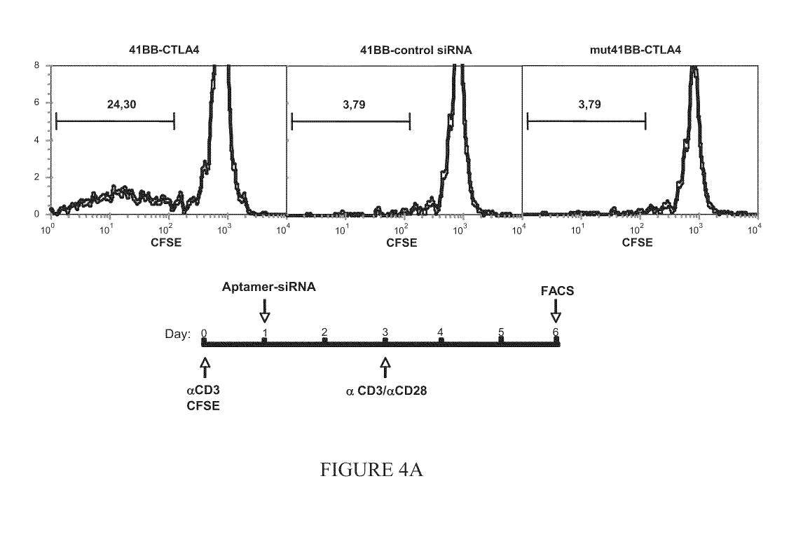

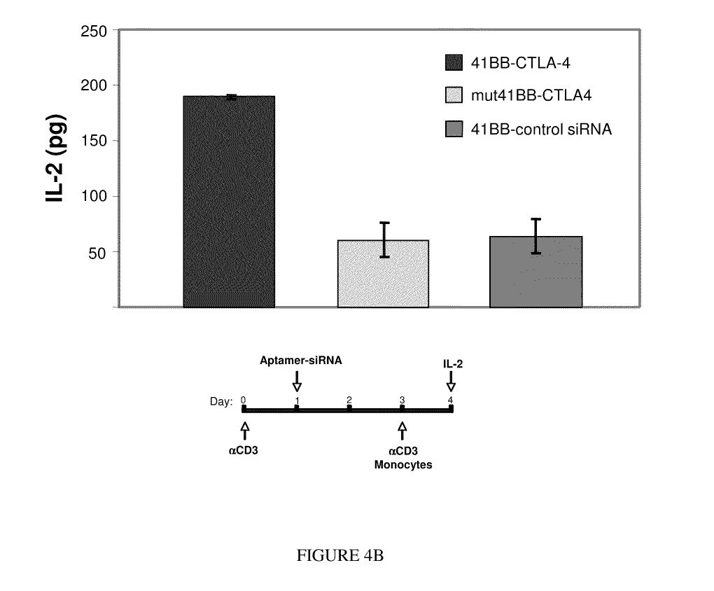

In a preferred embodiment, small interfering RNA (siRNA) either as RNA itself or as DNA, is delivered to a cell using aptamers. FIGS. 2A and 2B provide a schematic illustration of aptamer targeted siRNAs. Many different permutations and combinations of aptamers and RNAi's can be used. For example, the siRNA can be attached to one or more aptamers or encoded as a single molecule so that the 5′ to 3′ would encode for an aptamer, the siRNA and an aptamer. These can also be attached via linker molecules. The composition can also comprise in a 5′ to 3′ direction an aptamer attached to another aptamer via a linker which are then attached to the siRNA. These molecules can also be encoded in the same combination. Compositions can include various permutations and combinations. The composition can include siRNAs specific for different polynucleotide targets.

In certain embodiments, the nucleic acid molecules of the present disclosure can be synthesized separately and joined together post-synthetically, for example, by ligation (Moore et al., Science 256:9923, 1992; Draper et al., PCT Publication No. WO 93/23569; Shabarova et al., Nucleic Acids Res. 19:4247, 1991; Bellon et al., Nucleosides & Nucleotides 16:951, 1997; Bellon et al., Bioconjugate Chem. 8:204, 1997), or by hybridization following synthesis or deprotection.

In further embodiments, RNAi's can be made as single or multiple transcription products expressed by a polynucleotide vector encoding one or more siRNAs and directing their expression within host cells. An RNAi or analog thereof of this disclosure may be further comprised of a nucleotide, non-nucleotide, or mixed nucleotide/non-nucleotide linker that joins the aptamers and RNAi's. In one embodiment, a nucleotide linker can be a linker of more than about 2 nucleotides length up to about 50 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, e.g., Gold et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993, and Biochemistry 32:1751, 1993; Durand et al., Nucleic Acids Res. 18:6353, 1990; McCurdy et al., Nucleosides & Nucleotides 10:287, 1991; Jaschke et al., Tetrahedron Lett. 34:301, 1993; Ono et al., Biochemistry 30:9914, 1991; Arnold et al., PCT Publication No. WO 89/02439; Usman et al., PCT Publication No. WO 95/06731; Dudycz et al., PCT Publication No. WO 95/11910 and Ferentz and Verdine, J. Am. Chem. Soc. 113:4000, 1991.

The invention may be used against protein coding gene products as well as non-protein coding gene products. Examples of non-protein coding gene products include gene products that encode ribosomal RNAs, transfer RNAs, small nuclear RNAs, small cytoplasmic RNAs, telomerase RNA, RNA molecules involved in DNA replication, chromosomal rearrangement and the like.

In accordance with the invention, siRNA oligonucleotide therapies comprise administered siRNA oligonucleotide which contacts (interacts with) the targeted mRNA from the gene, whereby expression of the gene is modulated. Such modulation of expression suitably can be a difference of at least about 10% or 20% relative to a control, more preferably at least about 30%, 40%, 50%, 60%, 70%, 80%, or 90% difference in expression relative to a control. It will be particularly preferred where interaction or contact with an siRNA oligonucleotide results in complete or essentially complete modulation of expression relative to a control, e.g., at least about a 95%, 97%, 98%, 99% or 100% inhibition of or increase in expression relative to control. A control sample for determination of such modulation can be comparable cells (in vitro or in vivo) that have not been contacted with the siRNA oligonucleotide.

In another preferred embodiment, the nucleobases in the siRNA may be modified to provided higher specificity and affinity for a target mRNA. For example nucleobases may be substituted with LNA monomers, which can be in contiguous stretches or in different positions. The modified siRNA, preferably has a higher association constant (Kₐ) for the target sequences than the complementary sequence. Binding of the modified or non-modified siRNA's to target sequences can be determined in vitro under a variety of stringency conditions using hybridization assays and as described in the examples which follow.

A fundamental property of oligonucleotides that underlies many of their potential therapeutic applications is their ability to recognize and hybridize specifically to complementary single stranded nucleic acids employing either Watson-Crick hydrogen bonding (A-T and G-C) or other hydrogen bonding schemes such as the Hoögsteen/reverse Hoögsteen mode. Affinity and specificity are properties commonly employed to characterize hybridization characteristics of a particular oligonucleotide. Affinity is a measure of the binding strength of the oligonucleotide to its complementary target (expressed as the thermostability (Tₘ) of the duplex). Each nucleobase pair in the duplex adds to the thermostability and thus affinity increases with increasing size (No. of nucleobases) of the oligonucleotide. Specificity is a measure of the ability of the oligonucleotide to discriminate between a fully complementary and a mismatched target sequence. In other words, specificity is a measure of the loss of affinity associated with mismatched nucleobase pairs in the target.

The utility of an siRNA oligonucleotide for modulation (including inhibition) of an mRNA can be readily determined by simple testing. Thus, an in vitro or in vivo expression system comprising the targeted mRNA, mutations or fragments thereof, can be contacted with a particular siRNA oligonucleotide (modified or un modified) and levels of expression are compared to a control, that is, using the identical expression system which was not contacted with the siRNA oligonucleotide.

Aptamer-siRNA oligonucleotides may be used in combinations. For instance, a cocktail of several different siRNA modified and/or unmodified oligonucleotides, directed against different regions of the same gene, may be administered simultaneously or separately.

In the practice of the present invention, target gene products may be single-stranded or double-stranded DNA or RNA. Short dsRNA can be used to block transcription if they are of the same sequence as the start site for transcription of a particular gene. See, for example, Janowski et al. Nature Chemical Biology, 2005, 10:1038. It is understood that the target to which the siRNA oligonucleotides of the invention are directed include allelic forms of the targeted gene and the corresponding mRNAs including splice variants. There is substantial guidance in the literature for selecting particular sequences for siRNA oligonucleotides given a knowledge of the sequence of the target polynucleotide. Preferred mRNA targets include the 5′ cap site, tRNA primer binding site, the initiation codon site, the mRNA donor splice site, and the mRNA acceptor splice site.

Where the target polynucleotide comprises a mRNA transcript, sequence complementary oligonucleotides can hybridize to any desired portion of the transcript. Such oligonucleotides are, in principle, effective for inhibiting translation, and capable of inducing the effects described herein. It is hypothesized that translation is most effectively inhibited by the mRNA at a site at or near the initiation codon. Thus, oligonucleotides complementary to the 5′-region of mRNA transcript are preferred. Oligonucleotides complementary to the mRNA, including the initiation codon (the first codon at the 5′ end of the translated portion of the transcript), or codons adjacent to the initiation codon, are preferred.

Chimeric/modified RNAi's: In accordance with this invention, persons of ordinary skill in the art will understand that mRNA includes not only the coding region which carries the information to encode a protein using the three letter genetic code, including the translation start and stop codons, but also associated ribonucleotides which form a region known to such persons as the 5′-untranslated region, the 3′-untranslated region, the 5′ cap region, intron regions and intron/exon or splice junction ribonucleotides. Thus, oligonucleotides may be formulated in accordance with this invention which are targeted wholly or in part to these associated ribonucleotides as well as to the coding ribonucleotides. In preferred embodiments, the oligonucleotide is targeted to a translation initiation site (AUG codon) or sequences in the coding region, 5′ untranslated region or 3′-untranslated region of an mRNA. The functions of messenger RNA to be interfered with include all vital functions such as translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with protein expression.

Certain preferred oligonucleotides of this invention are chimeric oligonucleotides. “Chimeric oligonucleotides” or “chimeras,” in the context of this invention, are oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the RNA target) and a region that is a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art. In one preferred embodiment, a chimeric oligonucleotide comprises at least one region modified to increase target binding affinity, and, usually, a region that acts as a substrate for RNAse H. Affinity of an oligonucleotide for its target (in this case, a nucleic acid encoding ras) is routinely determined by measuring the Tₘ of an oligonucleotide/target pair, which is the temperature at which the oligonucleotide and target dissociate; dissociation is detected spectrophotometrically. The higher the Tₘ, the greater the affinity of the oligonucleotide for the target.

In another preferred embodiment, the region of the oligonucleotide which is modified comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrymidines, abasic residues or an inverted base at the 3′ end of the RNA. Such modifications are routinely incorporated into oligonucleotides and these oligonucleotides have been shown to have a higher Tₘ (i.e., higher target binding affinity) than; 2′-deoxyoligonucleotides against a given target. The effect of such increased affinity is to greatly enhance RNAi oligonucleotide inhibition of gene expression. RNAse H is a cellular endonuclease that cleaves the RNA strand of RNA:DNA duplexes; activation of this enzyme therefore results in cleavage of the RNA target, and thus can greatly enhance the efficiency of RNAi inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide.

Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. Oligonucleotides which contain at least one phosphorothioate modification are presently more preferred. In some cases, oligonucleotide modifications which enhance target binding affinity are also, independently, able to enhance nuclease resistance. Some desirable modifications can be found in De Mesmaeker et al. Acc. Chem. Res. 1995, 28:366-374.

Specific examples of some preferred oligonucleotides envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). Oligonucleotides may also comprise one or more substituted sugar moieties. Preferred oligonucleotides comprise one of the following at the 2′ position: OH, SH, SCH₃, F, OCN, OCH₃ OCH₃, OCH₃O(CH₂)ₙCH₃, O(CH₂)ₙNH₂ or O(CH₂)ₙCH₃ where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or N-alkyl; O, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂ CH₂ OCH₃, also known as 2′-O-(2-methoxyethyl)] (Martin et al., Helv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-5-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651). Oligonucleotides comprising lipophilic moieties, and methods for preparing such oligonucleotides are known in the art, for example, U.S. Pat. Nos. 5,138,045, 5,218,105 and 5,459,255.

It is not necessary for all positions in a given oligonucleotide to be uniformly modified, and in fact more than one of the aforementioned modifications may be incorporated in a single oligonucleotide or even at within a single nucleoside within an oligonucleotide. The present invention also includes oligonucleotides which are chimeric oligonucleotides as hereinbefore defined.

In another embodiment, the nucleic acid molecule of the present invention is conjugated with another moiety including but not limited to abasic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of one of ordinary skill in the art. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

In accordance with the invention, use of modifications such as the use of LNA monomers to enhance the potency, specificity and duration of action and broaden the routes of administration of oligonucleotides comprised of current chemistries such as MOE, ANA, FANA, PS etc (Recent advances in the medical chemistry of antisense oligonucleotide by Uhlman, Current Opinions in Drug Discovery & Development 2000 Vol 3 No 2). This can be achieved by substituting some of the monomers in the current oligonucleotides by LNA monomers. The LNA modified oligonucleotide may have a size similar to the parent compound or may be larger or preferably smaller. It is preferred that such LNA-modified oligonucleotides contain less than about 70%, more preferably less than about 60%, most preferably less than about 50% LNA monomers and that their sizes are between about 10 and 25 nucleotides, more preferably between about 12 and 20 nucleotides.

In a preferred embodiment, siRNA's target genes that prevent the normal expression or, if desired, over expression of genes that are of therapeutic interest as described above. As used herein, the term “overexpressing” when used in reference to the level of a gene expression is intended to mean an increased accumulation of the gene product in the overexpressing cells compared to their levels in counterpart normal cells. Overexpression can be achieved by natural biological phenomenon as well as by specific modifications as is the case with genetically engineered cells. Overexpression also includes the achievement of an increase in cell survival polypeptide by either endogenous or exogenous mechanisms. Overexpression by natural phenomenon can result by, for example, a mutation which increases expression, processing, transport, translation or stability of the RNA as well as mutations which result in increased stability or decreased degradation of the polypeptide. Such examples of increased expression levels are also examples of endogenous mechanisms of overexpression. A specific example of a natural biologic phenomenon which results in overexpression by exogenous mechanisms is the adjacent integration of a retrovirus or transposon. Overexpression by specific modification can be achieved by, for example, the use of siRNA oligonucleotides described herein.

An siRNA polynucleotide may be constructed in a number of different ways provided that it is capable of interfering with the expression of a target protein. The siRNA polynucleotide generally will be substantially identical (although in a complementary orientation) to the target molecule sequence. The minimal identity will typically be greater than about 80%, greater than about 90%, greater than about 95% or about 100% identical.

### Generation of Aptamers

Aptamers are high affinity single-stranded nucleic acid ligands which can be isolated from combinatorial libraries through an iterative process of in vitro selection known as SELEX™ (Systemic Evolution of Ligands by EXponential enrichment). Aptamers exhibit specificity and avidity comparable to or exceeding that of antibodies, and can be generated against most targets. Unlike antibodies, aptamers, or in this instance aptamer-siRNA fusions, can be synthesized in a chemical process and hence offer significant advantages in terms of reduced production cost and much simpler regulatory approval process. Also, aptamers-siRNAs are not expected to exhibit significant immunogenicity in vivo.

In preferred embodiments, the siRNA is linked to at least one aptamer which is specific for a desired cell and target molecule. In other embodiments, the RNAi's are combined with two aptamers. For example, FIG. 2B. The various permutations and combinations for combining aptamers and RNAi's is limited only by the imagination of the user.

Methods of the present disclosure do not require a priori knowledge of the nucleotide sequence of every possible gene variant (including mRNA splice variants) targeted by the RNAi or analog thereof.

Aptamers specific for a given biomolecule can be identified using techniques known in the art. See, e.g., Toole et al. (1992) PCT Publication No. WO 92/14843; Tuerk and Gold (1991) PCT Publication No. WO 91/19813; Weintraub and Hutchinson (1992) PCT Publication No. 92/05285; and Ellington and Szostak, Nature 346:818 (1990). Briefly, these techniques typically involve the complexation of the molecular target with a random mixture of oligonucleotides. The aptamer-molecular target complex is separated from the uncomplexed oligonucleotides. The aptamer is recovered from the separated complex and amplified. This cycle is repeated to identify those aptamer sequences with the highest affinity for the molecular target.

The SELEX™ process is a method for the in vitro evolution of nucleic acid molecules with highly specific binding to target molecules and is described in, e.g., U.S. Pat. No. 5,270,163 (see also WO 91/19813) entitled “Nucleic Acid Ligands”. Each SELEX-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotides comprising randomized sequences derived from chemical synthesis on a standard DNA synthesizer. The oligonucleotides can be modified or unmodified DNA, RNA or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a pre-selected purpose such as, CpG motifs, hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g., U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 10¹⁴-10¹⁶ individual molecules, a number sufficient for most SELEX™ experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be either RNA or DNA. In those instances where an RNA library is to be used as the starting library it is typically generated by transcribing a DNA library in vitro using T7 RNA polymerase or modified T7 RNA polymerases and purified. The RNA or DNA library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 10¹⁴ different nucleic acid species but may be used to sample as many as about 10¹⁸ different nucleic acid species. Generally, nucleic acid aptamer molecules are selected in a 5 to 20 cycle procedure. In one embodiment, heterogeneity is introduced only in the initial selection stages and does not occur throughout the replicating process. In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20 to about 50 nucleotides and in some embodiments, about 30 to about 40 nucleotides. In one example, the 5′-fixed:random:3′-fixed sequence comprises a random sequence of about 30 to about 50 nucleotides.

The core SELEX™ method can be modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photo reactive groups capable of binding and/or photo-cross linking to and/or photo-inactivating a target molecule. U.S. Pat. No. 5,567,588 and U.S. Pat. No. 5,861,254 describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target. SELEX™ can also be used to obtain nucleic acid ligands that bind to more than one site on the target molecule, and to obtain nucleic acid ligands that include non-nucleic acid species that bind to specific sites on the target.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; (e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX™, cycles of selection and amplification are repeated as necessary until a desired goal is achieved.

One potential problem encountered in the use of nucleic acids as therapeutics and vaccines is that oligonucleotides in their phosphodiester form may be quickly degraded in body fluids by intracellular and extracellular enzymes such as endonucleases and exonuclease before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. For example, oligonucleotides containing nucleotide derivatives chemically modified at the 2′ position of ribose, 5 position of pyrimidines, and 8 position of purines, 2′-modified pyrimidines, nucleotides modified with 2′-amino (2′—NH₂), 2′-fluoro (2′-F), and/or 2′-O-methyl (2′-OMe) substituents.

In preferred embodiments, one or more modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, and unusual base-pairing combinations such as the isobases isocytidine and isoguanosine. Modifications can also include 3′ and 5′ modifications such as capping.

In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atom.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX™ process modifications or post-SELEX™ process modifications (modification of previously identified unmodified ligands) or may be made by incorporation into the SELEX™ process.

Pre-SELEX™ process modifications or those made by incorporation into the SELEX™ process yield nucleic acid ligands with both specificity for their SELEX™ target and improved stability, e.g., in vivo stability. Post-SELEX™ process modifications made to nucleic acid ligands may result in improved stability, e.g., in vivo stability without adversely affecting the binding capacity of the nucleic acid ligand.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

The identification of nucleic acid ligands to small, flexible peptides via the SELEX™ method can also be used in embodiments of the invention. Small peptides have flexible structures and usually exist in solution in an equilibrium of multiple conformers.

The aptamers with specificity and binding affinity to the target(s) of the present invention are typically selected by the SELEX™ process as described herein. As part of the SELEX™ process, the sequences selected to bind to the target can then optionally be minimized to determine the minimal sequence having the desired binding affinity. The selected sequences and/or the minimized sequences are optionally optimized by performing random or directed mutagenesis of the sequence to increase binding affinity or alternatively to determine which positions in the sequence are essential for binding activity. Additionally, selections can be performed with sequences incorporating modified nucleotides to stabilize the aptamer molecules against degradation in vivo.

The results show that the aptamer-RNAi compositions enter cells and sub-cellular compartments. However, further aptamers can be obtained using various methods. In a preferred embodiment, a variation of the SELEX™ process is used to discover aptamers that are able to enter cells or the sub-cellular compartments within cells. These delivery aptamers will allow or increase the propensity of an oligonucleotide to enter or be taken up by a cell. The method comprises the ability to selectively amplify aptamers that have been exposed to the interior of a cell and became modified in some fashion as a result of that exposure. Such modifications include functioning as a template for template-dependent polymerization. This variation of SELEX™ permits the discovery of aptamers that are: (i) completely specific with regard to the kind of cell or sub-cellular compartment, such as the nucleus or cytoplasm, that they permit entry to, (ii) completely generic, or (iii) partially specific.

One potential strategy is to substitute cell-association for cell entry, and after incubation of the library with the cells and subsequent washing of the cells, amplify the library members that remain associated with the cells. However, this may not distinguish between aptamers that permit genuine cell entry and other trivial solutions to the cell-association problem such as binding to the exterior of the cell membrane, entering, but not leaving, the cell membrane and being taken up by, but not leaving, the endosome.

An alternative strategy is to select for some kind of transformation of the oligonucleotide library member that could happen only in the cytoplasm or other sub-cellular compartment, optionally because the library member is conjugated to a transformable entity, and then selectively amplifying the transformed library members. Such markers include, but are not limited to: reverse transcription, RNaseH, kinase, 5′-phosphorylation, 5′-dephosphorylation, translation-dependent, post-transcriptional modification to give restrictable cDNA, transcription-based, ubiquitination, ultracentrifugation, or utilizing the endogenous protein kinase Clp1. For example, library members can have a designed hairpin structure at their 3′-terminus that will reverse-transcribe without a primer. Reverse transcriptase activity is introduced into the cytoplasm using a protein expression vector or virus. The selective amplification of reverse-transcribed sequences is achieved by using a nucleotide composition that will not amplify directly by, for example, PCR such as completely or partially 2′-OH or 2′OMe RNA and omitting an RT step from the procedure.

### Identification of Target Nucleic Acid Sequences

With an emerging functional RNA world, there are new potential targets to be considered. Among these are large numbers of natural occurring antisense transcripts with a capacity to regulate the expression of sense transcripts including those that encode for conventional drug targets.

In a preferred embodiment, the compositions of the invention target desired nucleic acid sequences. Any desired target nucleic acid sequences can be identified by a variety of methods such as SAGE. SAGE is based on several principles. First, a short nucleotide sequence tag (9 to 10 b.p.) contains sufficient information content to uniquely identify a transcript provided it is isolated from a defined position within the transcript. For example, a sequence as short as 9 b.p. can distinguish 262,144 transcripts given a random nucleotide distribution at the tag site, whereas estimates suggest that the human genome encodes about 80,000 to 200,000 transcripts (Fields, et al., Nature Genetics, 7:345 1994). The size of the tag can be shorter for lower eukaryotes or prokaryotes, for example, where the number of transcripts encoded by the genome is lower. For example, a tag as short as 6-7 b.p. may be sufficient for distinguishing transcripts in yeast.

Second, random dimerization of tags allows a procedure for reducing bias (caused by amplification and/or cloning). Third, concatenation of these short sequence tags allows the efficient analysis of transcripts in a serial manner by sequencing multiple tags within a single vector or clone. As with serial communication by computers, wherein information is transmitted as a continuous string of data, serial analysis of the sequence tags requires a means to establish the register and boundaries of each tag. The concept of deriving a defined tag from a sequence in accordance with the present invention is useful in matching tags of samples to a sequence database. In the preferred embodiment, a computer method is used to match a sample sequence with known sequences.

The tags are used to uniquely identify gene products. This is due to their length, and their specific location (3′) in a gene from which they are drawn. The full length gene products can be identified by matching the tag to a gene data base member, or by using the tag sequences as probes to physically isolate previously unidentified gene products from cDNA libraries. The methods by which gene products are isolated from libraries using DNA probes are well known in the art. See, for example, Veculescu et al., Science 270: 484 (1995), and Sambrook et al. (1989), MOLECULAR CLONING: A LABORATORY MANUAL, 2nd ed. (Cold Spring Harbor Press, Cold Spring Harbor, N.Y.). Once a gene or transcript has been identified, either by matching to a data base entry, or by physically hybridizing to a cDNA molecule, the position of the hybridizing or matching region in the transcript can be determined. If the tag sequence is not in the 3′ end, immediately adjacent to the restriction enzyme used to generate the SAGE tags, then a spurious match may have been made. Confirmation of the identity of a SAGE tag can be made by comparing transcription levels of the tag to that of the identified gene in certain cell types.

Analysis of gene expression is not limited to the above methods but can include any method known in the art. All of these principles may be applied independently, in combination, or in combination with other known methods of sequence identification.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (Comb. Chem. High Throughput Screen, 2000, 3, 235-41)).

In yet another aspect, siRNA oligonucleotides that selectively bind to variants of target gene expression products. A “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet, at, for example, ncbi.nlm.nih.gov/BLAST/. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

In accordance with the invention, paralogs can be identified for designing the appropriate siRNA oligonucleotide. Paralogs are genes within a species that occur due to gene duplication, but have evolved new functions, and are also referred to as isotypes.

The polynucleotides of this invention can be isolated using the technique described in the experimental section or replicated using PCR. The PCR technology is the subject matter of U.S. Pat. Nos. 4,683,195, 4,800,159, 4,754,065, and 4,683,202 and described in PCR: The Polymerase Chain Reaction (Mullis et al. eds, Birkhauser Press, Boston (1994)) and references cited therein. Alternatively, one of skill in the art can use the identified sequences and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

Another suitable method for identifying targets for the aptamer-RNAi compositions includes contacting a test sample with a cell expressing a receptor or gene thereof, an allele or fragment thereof; and detecting interaction of the test sample with the gene, an allele or fragment thereof, or expression product of the gene, an allele or fragment thereof. The desired gene, an allele or fragment thereof, or expression product of the gene, an allele or fragment thereof suitably can be detectably labeled e.g. with a fluorescent or radioactive component.

In another preferred embodiment, a cell from a patient is isolated and contacted with a drug molecule that modulates an immune response. The genes, expression products thereof, are monitored to identify which genes or expression products are regulated by the drug. Interference RNA's can then be synthesized to regulate the identified genes, expression products that are regulated by the drug and thus, provide therapeutic oligonucleotides. These can be tailored to individual patients, which is advantageous as different patients do not effectively respond to the same drugs equally. Thus, the oligonucleotides would provide a cheaper and individualized treatment than conventional drug treatments.

In one aspect, hybridization with oligonucleotide probes that are capable of detecting polynucleotide sequences, including genomic sequences, encoding desired genes or closely related molecules may be used to identify target nucleic acid sequences. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding genes, allelic variants, or related sequences.

Probes may also be used for the detection of related sequences, and should preferably have at least 50% sequence identity or homology to any of the identified genes encoding sequences, more preferably at least about 60, 70, 75, 80, 85, 90 or 95 percent sequence identity to any of the identified gene encoding sequences (sequence identity determinations discussed above, including use of BLAST program). The hybridization probes of the subject invention may be DNA or RNA and may be derived from the sequences of the invention or from genomic sequences including promoters, enhancers, and introns of the gene.

“Homologous,” as used herein, refers to the subunit sequence similarity between two polymeric molecules, e.g., between two nucleic acid molecules such as two DNA molecules, or two polypeptide molecules. When a subunit position in both of the two molecules is occupied by the same monomeric subunit (e.g., if a position in each of two DNA molecules is occupied by adenine) then they are homologous at that position. The homology between two sequences is a direct function of the number of matching or homologous positions. For example, if 5 of 10 positions in two compound sequences are matched or homologous then the two sequences are 50% homologous, if 9 of 10 are matched or homologous, the two sequences share 90% homology. By way of example, the DNA sequences 3′ ATTGCC 5′ and 3′ TTTCCG 5′ share 50% homology.

Means for producing specific hybridization probes for polynucleotides encoding target genes include the cloning of polynucleotide sequences encoding target genes or derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³²S, or by enzymatic labels, such as alkaline phosphatase coupled to the probe via avidin-biotin coupling systems, fluorescent labeling, and the like.

The polynucleotide sequences encoding a target gene may be used in Southern or Northern analysis, dot blot, or other membrane-based technologies; in PCR technologies; in dipstick, pin, and multiformat ELISA-like assays; and in microarrays utilizing fluids or tissues from patients to detect altered target gene expression. Gel-based mobility-shift analyses may be employed. Other suitable qualitative or quantitative methods are well known in the art.

Identity of genes, or variants thereof, can be verified using techniques well known in the art. Examples include but are not limited to, nucleic acid sequencing of amplified genes, hybridization techniques such as single nucleic acid polymorphism analysis (SNP), microarrays wherein the molecule of interest is immobilized on a biochip. Overlapping cDNA clones can be sequenced by the dideoxy chain reaction using fluorescent dye terminators and an ABI sequencer (Applied Biosystems, Foster City, Calif.). Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

An assay using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Additional diagnostic uses for oligonucleotides designed from the sequences encoding a desired gene expression product may involve the use of PCR. These oligomers may be chemically synthesized, generated enzymatically, or produced in vitro. Oligomers will preferably contain a fragment of a polynucleotide encoding the expression products, or a fragment of a polynucleotide complementary to the polynucleotides, and will be employed under optimized conditions for identification of a specific gene. Oligomers may also be employed under less stringent conditions for detection or quantitation of closely-related DNA or RNA sequences.

In further embodiments, oligonucleotides or longer fragments derived from any of the polynucleotide sequences, may be used as targets in a microarray. The microarray can be used to monitor the identity and/or expression level of large numbers of genes and gene transcripts simultaneously to identify genes with which target genes or its product interacts and/or to assess the efficacy of candidate aptamer-RNAi compositions in regulating expression products of genes that mediate, for example, tumor specific immune responses. This information may be used to determine gene function, and to develop and monitor the activities of compositions.

Microarrays may be prepared, used, and analyzed using methods known in the art (see, e.g., Brennan et al., 1995, U.S. Pat. No. 5,474,796; Schena et al., 1996, Proc. Natl. Acad. Sci. U.S.A. 93: 10614-10619; Baldeschweiler et al., 1995, PCT application WO95/251116; Shalon, et al., 1995, PCT application WO95/35505; Heller et al., 1997, Proc. Natl. Acad. Sci. U.S.A. 94: 2150-2155; and Heller et al., 1997, U.S. Pat. No. 5,605,662).

In other preferred embodiments, high throughput screening (HTS) can be used to measure the effects of RNAi's on complex molecular events such as signal transduction pathways, as well as cell functions including, but not limited to, cell function, apoptosis, cell division, cell adhesion, locomotion, exocytosis, and cell-cell communication. Multicolor fluorescence permits multiple targets and cell processes to be assayed in a single screen. Cross-correlation of cellular responses will yield a wealth of information required for target validation and lead optimization.

In another aspect, the present invention provides a method for analyzing cells comprising providing an array of locations which contain multiple cells wherein the cells contain one or more fluorescent reporter molecules; scanning multiple cells in each of the locations containing cells to obtain fluorescent signals from the fluorescent reporter molecule in the cells; converting the fluorescent signals into digital data; and utilizing the digital data to determine the distribution, environment or activity of the fluorescent reporter molecule within the cells.

A major component of the new drug discovery paradigm is a continually growing family of fluorescent and luminescent reagents that are used to measure the temporal and spatial distribution, content, and activity of intracellular ions, metabolites, macromolecules, and organelles. Classes of these reagents include labeling reagents that measure the distribution and amount of molecules in living and fixed cells, environmental indicators to report signal transduction events in time and space, and fluorescent protein biosensors to measure target molecular activities within living cells. A multiparameter approach that combines several reagents in a single cell is a powerful new tool for drug discovery.

This method relies on the high affinity of fluorescent or luminescent molecules for specific cellular components. The affinity for specific components is governed by physical forces such as ionic interactions, covalent bonding (which includes chimeric fusion with protein-based chromophores, fluorophores, and lumiphores), as well as hydrophobic interactions, electrical potential, and, in some cases, simple entrapment within a cellular component. The luminescent probes can be small molecules, labeled macromolecules, or genetically engineered proteins, including, but not limited to green fluorescent protein chimeras.

Those skilled in this art will recognize a wide variety of fluorescent reporter molecules that can be used in the present invention, including, but not limited to, fluorescently labeled biomolecules such as proteins, phospholipids, RNA and DNA hybridizing probes. Similarly, fluorescent reagents specifically synthesized with particular chemical properties of binding or association have been used as fluorescent reporter molecules (Barak et al., (1997), J. Biol. Chem. 272:27497-27500; Southwick et al., (1990), Cytometry 11:418-430; Tsien (1989) in Methods in Cell Biology, Vol. 29 Taylor and Wang (eds.), pp. 127-156). Fluorescently labeled antibodies are particularly useful reporter molecules due to their high degree of specificity for attaching to a single molecular target in a mixture of molecules as complex as a cell or tissue.

The luminescent probes can be synthesized within the living cell or can be transported into the cell via several non-mechanical modes including diffusion, facilitated or active transport, signal-sequence-mediated transport, and endocytotic or pinocytotic uptake. Mechanical bulk loading methods, which are well known in the art, can also be used to load luminescent probes into living cells (Barber et al. (1996), Neuroscience Letters 207:17-20; Bright et al. (1996), Cytometry 24:226-233; McNeil (1989) in Methods in Cell Biology, Vol. 29, Taylor and Wang (eds.), pp. 153-173). These methods include electroporation and other mechanical methods such as scrape-loading, bead-loading, impact-loading, syringe-loading, hypertonic and hypotonic loading. Additionally, cells can be genetically engineered to express reporter molecules, such as GFP, coupled to an RNAi or probes of interest.

Once in the cell, the luminescent probes accumulate at their target domain as a result of specific and high affinity interactions with the target domain or other modes of molecular targeting such as signal-sequence-mediated transport. Fluorescently labeled reporter molecules are useful for determining the location, amount and chemical environment of the reporter. For example, whether the reporter is in a lipophilic membrane environment or in a more aqueous environment can be determined (Giuliano et al. (1995), Ann. Rev. of Biophysics and Biomolecular Structure 24:405-434; Giuliano and Taylor (1995), Methods in Neuroscience 27.1-16). The pH environment of the reporter can be determined (Bright et al. (1989), J. Cell Biology 104:1019-1033; Giuliano et al. (1987), Anal. Biochem. 167:362-371; Thomas et al. (1979), Biochemistry 18:2210-2218). It can be determined whether a reporter having a chelating group is bound to an ion, such as Ca⁺⁺, or not (Bright et al. (1989), In Methods in Cell Biology, Vol. 30, Taylor and Wang (eds.), pp. 157-192; Shimoura et al. (1988), J. of Biochemistry (Tokyo) 251:405-410; Tsien (1989) In Methods in Cell Biology, Vol. 30, Taylor and Wang (eds.), pp. 127-156).

Those skilled in the art will recognize a wide variety of ways to measure fluorescence. For example, some fluorescent reporter molecules exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements (Giuliano et al. (1995), Ann. Rev. of Biophysics and Biomol. Structure 24:405-434; Giuliano et al. (1995), Methods in Neuroscience 27:1-16).

The whole procedure can be fully automated. For example, sampling of sample materials may be accomplished with a plurality of steps, which include withdrawing a sample from a sample container and delivering at least a portion of the withdrawn sample to test cell culture (e.g., a cell culture wherein gene expression is regulated). Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples from a process control line). Preferably, however, the sample(s) are withdrawn from a sample container and delivered to the characterization system, in a fully automated manner—for example, with an auto-sampler.

In one embodiment, auto-sampling may be done using a microprocessor controlling an automated system (e.g., a robot arm). Preferably, the microprocessor is user-programmable to accommodate libraries of samples having varying arrangements of samples (e.g., square arrays with “n-rows” by “n-columns,” rectangular arrays with “n-rows” by “m-columns,” round arrays, triangular arrays with “r-” by “r-” by “r-” equilateral sides, triangular arrays with “r-base” by “s-” by “s-” isosceles sides, etc., where n, m, r, and s are integers).

Automated sampling of sample materials optionally may be effected with an auto-sampler having a heated injection probe (tip). An example of one such auto sampler is disclosed in U.S. Pat. No. 6,175,409 B1 (incorporated by reference).

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually.

### Assessing Up-Regulation or Inhibition of Gene Expression

Transfer of an exogenous nucleic acid into a host cell or organism can be assessed by directly detecting the presence of the nucleic acid in the cell or organism. Such detection can be achieved by several methods well known in the art. For example, the presence of the exogenous nucleic acid can be detected by Southern blot or by a polymerase chain reaction (PCR) technique using primers that specifically amplify nucleotide sequences associated with the nucleic acid. Expression of the exogenous nucleic acids can also be measured using conventional methods. For instance, mRNA produced from an exogenous nucleic acid can be detected and quantified using a Northern blot and reverse transcription PCR (RT-PCR).

Expression of an RNA from the exogenous nucleic acid can also be detected by measuring an enzymatic activity or a reporter protein activity. For example, siRNA activity can be measured indirectly as a decrease or increase in target nucleic acid expression as an indication that the exogenous nucleic acid is producing the effector RNA. Based on sequence conservation, primers can be designed and used to amplify coding regions of the target genes. Initially, the most highly expressed coding region from each gene can be used to build a model control gene, although any coding or non coding region can be used. Each control gene is assembled by inserting each coding region between a reporter coding region and its poly(A) signal. These plasmids would produce an mRNA with a reporter gene in the upstream portion of the gene and a potential RNAi target in the 3′ non-coding region. The effectiveness of individual RNAi's would be assayed by modulation of the reporter gene. Reporter genes useful in the methods of the present invention include acetohydroxy acid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline. Methods to determine modulation of a reporter gene are well known in the art, and include, but are not limited to, fluorometric methods (e.g. fluorescence spectroscopy, Fluorescence Activated Cell Sorting (FACS), fluorescence microscopy), antibiotic resistance determination.

Although biogenomic information and model genes are invaluable for high-throughput screening of potential RNAi's, interference activity against target nucleic acids ultimately must be established experimentally in cells which express the target nucleic acid. To determine the interference capability of the RNAi sequence, the RNAi containing vector is transfected into appropriate cell lines which express that target nucleic acid. Each selected RNAi construct is tested for its ability to modulate steady-state mRNA of the target nucleic acid. In addition, any target mRNAs that “survive” the first round of testing are amplified by reverse transcriptase-PCR and sequenced (see, for example, Sambrook, J. et al. “Molecular Cloning: A Laboratory Manual,” 2nd addition, Cold Spring Harbor Laboratory Press, Plainview, N.Y. (1989)). These sequences are analyzed to determine individual polymorphisms that allow mRNA to escape the current library of RNAi's. This information is used to further modify RNAi constructs to also target rarer polymorphisms.

Methods by which to transfect cells with RNAi vectors are well known in the art and include, but are not limited to, electroporation, particle bombardment, microinjection, transfection with viral vectors, transfection with retrovirus-based vectors, and liposome-mediated transfection. Any of the types of nucleic acids that mediate RNA interference can be synthesized in vitro using a variety of methods well known in the art and inserted directly into a cell. In addition, dsRNA and other molecules that mediate RNA interference are available from commercial vendors, such as Ribopharma AG (Kulmach, Germany), Eurogentec (Seraing, Belgium), Sequitur (Natick, Mass.) and Invitrogen (Carlsbad, Calif.). Eurogentec offers dsRNA that has been labeled with fluorophores (e.g., HEX/TET; 5′-Fluorescein, 6-FAM; 3′-Fluorescein, 6-FAM; Fluorescein dT internal; 5′ TAMRA, Rhodamine; 3′ TAMRA, Rhodamine), which can also be used in the invention. RNAi molecules can be made through the well-known technique of solid-phase synthesis. Equipment for such synthesis is sold by several vendors including, for example, Applied Biosystems (Foster City, Calif.). Other methods for such synthesis that are known in the art can additionally or alternatively be employed. It is well-known to use similar techniques to prepare oligonucleotides such as the phosphorothioates and alkylated derivatives.

RNA directly inserted into a cell can include modifications to either the phosphate-sugar backbone or the nucleoside. For example, the phosphodiester linkages of natural RNA can be modified to include at least one of a nitrogen or sulfur heteroatom. The interfering RNA can be produced enzymatically or by partial/total organic synthesis. The constructs can be synthesized by a cellular RNA polymerase or a bacteriophage RNA polymerase (e.g., T3, T7, SP6). If synthesized chemically or by in vitro enzymatic synthesis, the RNA can be purified prior to introduction into a cell or animal. For example, RNA can be purified from a mixture by extraction with a solvent or resin, precipitation, electrophoresis, chromatography or a combination thereof as known in the art. Alternatively, the interfering RNA construct can be used without, or with a minimum of purification to avoid losses due to sample processing. The RNAi construct can be dried for storage or dissolved in an aqueous solution. The solution can contain buffers or salts to promote annealing, and/or stabilization of the duplex strands. Examples of buffers or salts that can be used in the present invention include, but are not limited to, saline, PBS, N-(2-Hydroxyethyl)piperazin-e-N′-(2-ethanesulfonic acid) (HEPES™), 3-(N-Morpholino)propanesulfonic acid (MOPS), 2-bis(2-Hydroxyethylene)amino-2-(hydroxymethyl)-1,3-propaned-iol (bis-TRIS™), potassium phosphate (KP), sodium phosphate (NaP), dibasic sodium phosphate (Na₂HPO₄), monobasic sodium phosphate (NaH₂PO₄), monobasic sodium potassium phosphate (NaKHPO₄), magnesium phosphate (Mg₃(PO₄)₂₋₄H₂O), potassium acetate (CH3COOH), D(+)-α-sodium glycerophosphate (HOCH₂CH(OH)CH₂OPO₃Na₂) and other physiologic buffers known to those skilled in the art. Additional buffers for use in the invention include, a salt M-X dissolved in aqueous solution, association, or dissociation products thereof, where M is an alkali metal (e.g., Li⁺, Na⁺, K⁺, Rb⁺), suitably sodium or potassium, and where X is an anion selected from the group consisting of phosphate, acetate, bicarbonate, sulfate, pyruvate, and an organic monophosphate ester, glucose 6-phosphate or DL-α-glycerol phosphate.

### Genes Regulated/Targeted by RNAi Molecules.

In a further aspect of the present invention, RNAi molecules that regulate the expression of specific genes or family of genes are provided, such that the expression of the genes can be functionally eliminated or up-regulated. In one embodiment, at least two RNAi molecules are provided that target the same region of a gene. In another embodiment, at least two RNAi molecules are provided that target at least two different regions of the same gene. In a further embodiment, at least two RNAi molecules are provided that target at least two different genes. Additional embodiments of the invention provide combinations of the above strategies for gene targeting.

In another preferred embodiment, the aptamers and/or targeting agents are specific for different cell types or different cell-specific molecules or differing specificities on the same cell-specific molecule or combinations thereof. The number of targeting agents and specificities of each per chimeric molecule is limited only by the imagination of the user. The RNAi's can be specific for the same genes in these different cell types or different sequences in the same cell type and the like. Thus, in some embodiments, a cocktail of aptamer-RNAi's with differing specificities are provided. In another preferred embodiment, the chimeric molecule comprises one or more aptamers or targeting agents which can be specific for the same or different molecules. The chimeric molecule can also comprise one or more interference RNA molecules which specifically interfere with one or more target sequences. Thus the chimeric molecule can have one or more specificities and target molecules.

In one embodiment, the RNAi molecules can be the same sequence. In an alternate embodiment, the RNAi molecules can be different sequences. In other embodiments, at least two RNAi molecules are provided wherein the families of one or more genes can be regulated by expression of the RNAi molecules. In another embodiment, at least three, four or five RNAi molecules are provided wherein the families of one or more genes can be regulated (modulated) by expression of the RNAi molecules. The RNAi molecule can be homologous to a conserved sequence within one or more genes. The family of genes regulated using such methods of the invention can be endogenous to a cell, a family of related viral genes, a family of genes that are conserved within a viral genus, a family of related eukaryotic parasite genes, or more particularly a family of genes from a porcine endogenous retrovirus. In one specific embodiment, at least two RNAi molecules can target the at least two different genes, which are members of the same family of genes. The RNAi molecules can target homologous regions within a family of genes and thus one RNAi molecule can target the same region of multiple genes.

The RNAi molecule can be selected from, but not limited to the following types of RNAi: antisense oligonucleotides, ribozymes, small interfering RNAs (sRNAis), double stranded RNAs (dsRNAs), inverted repeats, short hairpin RNAs (shRNAs), small temporally regulated RNAs, and clustered inhibitory RNAs (cRNAis), including radial clustered inhibitory RNA, asymmetric clustered inhibitory RNA, linear clustered inhibitory RNA, and complex or compound clustered inhibitory RNA.

In another embodiment, expression of RNAi molecules for regulating target genes in mammalian cell lines or transgenic animals is provided such that expression of the target gene is functionally eliminated or below detectable levels or up-regulated, i.e. the expression of the target gene is decreased or increased by at least about 70%, 75%, 80%, 85%, 90%, 95%, 97% or 99%.

In another embodiment of this aspect of the present invention, methods are provided to produce cells and animals in which interfering RNA molecules are expressed to regulate the expression of target genes. Methods according to this aspect of the invention can comprise, for example: identifying one or more target nucleic acid sequences in a cell; obtaining at least one RNAi molecule that bind to the target nucleic acid sequence(s); introducing the RNAi molecules, optionally packaged in an expression vector, into the cell; and expressing the RNAi's in the cell under conditions such that the RNAi's bind to the target nucleic acid sequences, thereby regulating expression of one or more target genes.

In embodiments of the present invention, endogenous genes that can be regulated by the expression of at least one RNAi molecule include, but are not limited to, genes required for cell survival or cell replication, genes that encode an immunoglobulin locus, for example, Kappa light chain, and genes that encode a cell surface protein, for example, T cell receptor, co-stimulatory antigens and receptors, e.g. CD137, Vascular Cell Adhesion Molecule (VCAM) and other genes important to the structure and/or function of cells, tissues, organs and animals. The methods of the invention can also be used to regulate the expression of one or more non-coding RNA sequences. These non-coding RNA sequences can be sequences of an RNA virus genome, an endogenous gene, a eukaryotic parasite gene, or other non-coding RNA sequences that are known in the art and that will be familiar to the ordinarily skilled artisan. RNAi molecules that are expressed in cells or animals according to the aspects of the present invention can decrease, increase or maintain expression of one or more target genes. In order to identify specific target nucleic acid regions in which the expression of one or more genes, family of genes, desired subset of genes, or alleles of a gene is to be regulated, a representative sample of sequences for each target gene can be obtained. Sequences can be compared to find similar and dissimilar regions. This analysis can determine regions of identity between all family members and within subsets (i.e. groups within the gene family) of family members. In addition, this analysis can determines region of identity between alleles of each family member. By considering regions of identity between alleles of family members, between subsets of family members, and across the entire family, target regions can be identified that specify the entire family, subsets of family members, individual family members, subsets of alleles of individual family members, or individual alleles of family members.

Regulation (modulation) of expression can decrease expression of one or more target genes. Decreased expression results in post-transcriptional down-regulation of the target gene and ultimately the final product protein of the target gene. For down-regulation, the target nucleic acid sequences are identified such that binding of the RNAi to the sequence will decrease expression of the target gene. Decreased expression of a gene refers to the absence of, or observable or detectable decrease in, the level of protein and/or mRNA product from a target gene relative to that without the introduction of the RNAi. Complete suppression/inhibition as well as partial suppressed expression of the target gene are possible with the methods of the present invention. By “partial suppressed expression,” it is meant that the target gene is suppressed (i.e. the expression of the target gene is reduced) from about 10% to about 99%, with 100% being complete suppression/inhibition of the target gene. For example, about 10%, about 20%, about 30%, about 40%, about 50%, about 60%, about 70%, about 80%, about 90%, about 95%, or about 99% of gene expression of the one or more genes can be suppressed. Alternatively, expression is suppressed or inhibited below detectable threshold limits.

In other embodiments of the invention, regulation of expression can increase expression of one or more genes. Increased expression can result as discussed in detail in the examples which follow. In this embodiment of the invention, the target nucleic acid and the gene of interest can be separate sequences. Increased expression of a gene refers to the presence, or observable increase, in the level of protein and/or mRNA product from one or more target genes relative to that without the introduction of the RNAi. By increased expression of a gene, it is meant that the measurable amount of the target gene that is expressed is increased any amount relative to that without the introduction of the RNAi. For example, the level of expression of the gene can be increased about two-fold, about five-fold, about 10-fold, about 50-fold, about 100-fold, about 500-fold, about 1000-fold, or about 2000-fold, above that which occurs in the absence of the interfering RNA.

In still other aspects of the invention, regulation of expression can maintain expression of one or more genes, when the one or more genes are placed under environmental conditions that generally lead to increased or decreased expression of the one or more genes. Expression of one or more genes can be maintained under environmental conditions that would normally increase or decrease gene expression results in a steady-state level (i.e. no increase or decrease in expression with time) of gene expression relative to expression prior to the presence of environmental conditions that would otherwise increase or decrease expression. Examples of environmental conditions that can increase gene expression include, but are not limited to, the presence of growth factors, increased glucose production, hyperthermia and cell cycle changes. Examples of environmental conditions that can decrease gene expression include, but are not limited to, hypoxia, hypothermia, lack of growth factors and glucose depletion.

Quantitation of gene expression allows determination of the degree of inhibition (or enhancement) of gene expression in a cell or animal that contain one or more RNAi molecules. Lower doses of injected material and longer times after administration or integration of the RNAi can result in inhibition or enhancement in a smaller fraction of cells or animals (e.g., at least 10%, 20%, 50%, 75%, 90%, or 95% of targeted cells or animals). Quantitation of gene expression in a cell or animal can show similar amounts of inhibition or enhancement at the level of accumulation of target mRNA or translation of target protein. The efficiency of inhibition or enhancement can be determined by assessing the amount of gene product in the cell or animal using any method known in the art. For example, mRNA can be detected with a hybridization probe having a nucleotide sequence outside the region used for the interfering RNA, or translated polypeptide can be detected with an antibody raised against the polypeptide sequence of that region. Methods by which to quantitate mRNA and polypeptides are well-known in the art see, for example, Sambrook, J. et al. “Molecular Cloning: A Laboratory Manual,” 2nd addition, Cold Spring Harbor Laboratory Press, Plainview, N.Y. (1989).

As discussed above, the present invention also relates to the regulation of expression of a family of genes. The term “family of genes” refers to one or more genes that have a similar function, sequence, or phenotype. A family of genes can contain a conserved sequence, i.e. a nucleotide sequence that is the same or highly homologous among all members of the gene family. In certain embodiments, the RNAi sequence can hybridize to this conserved region of a gene family, and thus one RNAi sequence can target more than one member of a gene family.

The methods of the present invention can also be used to regulate expression of genes within an evolutionarily related family of genes. Evolutionarily related genes are genes that have diverged from a common progenitor genetic sequence, which can or can not have itself been a sequence encoding for one or more mRNAs. Within this evolutionarily related family can exist a subset of genes, and within this subset, a conserved nucleotide sequence can exist. The present invention also provides methods by which to regulate expression of this subset of genes by targeting the RNAi molecules to this conserved nucleotide sequence. Evolutionarily related genes that can be regulated by the methods of the present invention can be endogenous or exogenous to a cell or an animal and can be members of a viral family of genes. In addition, the family of viral genes that can be regulated by the methods of the present invention can have family members that are endogenous to the cell or animal.

In other embodiments, the methods of the present invention can be used to regulate expression of genes, or family of genes, that are endogenous to a cell or animal. An endogenous gene is any gene that is heritable as an integral element of the genome of the animal species. Regulation of endogenous genes by methods of the invention can provide a method by which to suppress or enhance a phenotype or biological state of a cell or an animal. Endogenous genes that can be regulated by the methods of the invention include, but are not limited to, endogenous genes that are required for T cell responses and the products of polynucleotides or polynucleotides associated with regulation of immune responses; endogenous genes that regulate cell survival; endogenous genes that are required for cell replication; endogenous genes that are required for viral replication; endogenous genes that encode an immunoglobulin locus, and endogenous genes that encode a cell surface protein.

Other endogenous genes include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin.

Further examples of endogenous genes include developmental genes (e.g., adhesion molecules, cyclin kinase inhibitors, Writ family members, Pax family members, Winged helix family members, Hox family members, cytokines/lymphokines and their receptors, growth/differentiation factors and their receptors, neurotransmitters and their receptors), tumor suppressor genes (e.g., APC, BRCA1, BRCA2, MADH4, MCC, NF 1, NF2, RB 1, TP53, and WTI) and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

In other embodiments, it may be desirable to regulate (modulate) tumor antigens in a cell so that, for example, these tumor cells can be detected by the host immune system. Many tumor antigens are well known in the art. See for example, Van den Eynde B J, van der Bruggen P. Curr Opin Immunol 1997; 9: 684-93; Houghton A N, Gold J S, Blachere N E. Curr Opin Immunol 2001; 13: 134-140; van der Bruggen P, Zhang Y, Chaux P, Stroobant V, Panichelli C, Schultz E S, Chapiro J, Van den Eynde B J, Bras seur F, Boon T. Immunol Rev 2002; 188: 51-64, which are herein incorporated by reference. Alternatively, many antibodies directed towards tumor antigens are commercially available.

In another preferred embodiment, a method of treating tumors comprises administering to a patient in need thereof a therapeutically effective chimeric molecule which specifically binds to immune cells or cells in tumor vasculatures. The chimeric molecule comprises at least one targeting agent which specifically binds to at least one target molecule and at least one interference RNA molecule which binds to at least one target sequence. Thus, the molecules can have more than one specificity by the targeting agents and/or the target sequence.

In another preferred embodiment, the targeting agent, for example, aptamer-siRNA are targeted to cells and molecules in diseases wherein immune cells are involved in the disease, such as autoimmune disease; hypersensitivity to allergens; organ rejection; inflammation; and the like. Examples of inflammation associated with conditions such as: adult respiratory distress syndrome (ARDS) or multiple organ injury syndromes secondary to septicemia or trauma; reperfusion injury of myocardial or other tissues; acute glomerulonephritis; reactive arthritis; dermatoses with acute inflammatory components; acute purulent meningitis or other central nervous system inflammatory disorders; thermal injury; hemodialysis; leukapheresis; ulcerative colitis; Crohn's disease; necrotizing enterocolitis; granulocyte transfusion associated syndromes; and cytokine-induced toxicity. Examples of autoimmune diseases include, but are not limited to psoriasis, Type I diabetes, Reynaud's syndrome, autoimmune thyroiditis, EAE, multiple sclerosis, rheumatoid arthritis and lupus erythematosus.

As an example, Tables 1 through 5 list a number of genes from which mRNA is transcribed, that may be modulated by siRNA or targeted by an aptamer; table 1 (CD markers), table 2 (adhesion molecules) table 3 (chemokines and chemokine receptors), table 4 (interleukins and their receptors) and table 5 (human non-CD antigens). Also included are the genes encoding the immunoglobulin E (IgE) and the IgE-receptor (FcεRIα) as well as the genes for the other immunoglobulins, IgG(₁₋₄), IgA₁, IgA₂, IgM, IgE, and IgD encoding free and membrane bound immunoglobulins and the genes encoding their corresponding receptors.

TABLE 1
CD markers

CD1a-d
CD2
CD3
CD4
CD5
CD6
CD7
CD8
CD9
CD10
CD11a
CD11b
CD11c
CDw12
CD13
CD14
CD15
CD16
CDw17
CD18
CD19
CD20
CD21
CD22
CD23
CD24
CD25
CD26
CD27
CD28
CD29
CD30
CD30
CD31
CD32
CD33
CD34
CD35
CD36
CD37
CD38
CD39
CD40
CD41
CD42a-d
CD43
CD44
CD45
CD46
CD47
CD48
CD49a-f
CD50
CD51
CD52
CD53
CD54
CD55
CD56
CD57
CD58
CD59
CDw60
CD61
CD62E
CD62L
CD62P
CD63
CD64
CD65
CD66a-e
CD67
CD68
CD69
CD70
CD71
CD72
CD73
CD74
CDw75
CDw76
CD77
CDw78
CD79a, b
CD80
CD81
CD82
CD83
CDw84
CD85
CD86
CD87
CD88
CD89
CD90
CD91
CDw92
CD93
CD94
CD95
CD96
CD97
CD98
CD99
CD100
CD101
CD102
CD103
CD104
CD105
CD106
CD107a, b
CDw08
CD109
CD110
CD111
CD112
CD113
CD114
CD115
CD116
CD117
CD118
CD119
CD120a, b
CD121
CD122
CDw123
CD124
CDw125
CD126
CD127
CDw128
CD129
CD130
CDw131
CD132
CD133
CD134
CD135
CD136
CD137
CD138 to CD339

TABLE 2
Adhesion molecules

| | | | | |
|---|---|---|---|---|
| L-selectin | TCRγ/δ | BB-1 | Integrin α7 | Integrin α6 |
| P-selectin | CD28 | N-cadherin | Integrin α8 | Integrin β5 |
| E-selectin | LFA-3 | E-cadherin | IntegrinαV | Integrin αV |
| HNK-1 | PECAM-1 | P-cadherin | Integrin β2 | Integrin β6 |
| Sialyl-Lewis X | VCAM-1 | Integrin β1 | Integrin αL | Integrin αV |
| CD15 | ICAM-2 | Integrin α1 | IntegrinαM | Integrin β7 |
| LFA-2 | ICAM-3 | Integrin α2 | IntegrinαX | IntegrinαIEL |
| CD22 | Leukosialin | Integrin α3 | Integrin β3 | Integrin α4 |
| ICAM-1 | HCAM | Integrin α4 | IntegrinαV | Integrin β8 |
| N-CAM | CD45RO | Integrin α5 | IntegrinαIib | Integrin αV |
| Ng-CAM | CD5 | Integrin α6 | Integrin β4 | |
| TCRα/β | HPCA-2 | | | |

TABLE 3
Chemokines and Chemokine receptors

| C—X—C | | | C | Chemokine |
|---|---|---|---|---|
| chemokines | C-C chemokines | | chemokines | Receptors |
| IL-8 | MCAF/MCP-1 | ABCD-1 | Lymphotactin | CCR1 |
| NAP-2 | MIP-1 α,β | LMC | | CCR2 |
| GRO/MGSA | RANTES | AMAC-1 | | CCR3 |
| γIP-10 | I-309 | NCC-4 | | CCR4 |
| ENA-78 | CCF18 | LKN-1 | | CCR5 |
| SDF-1 | SLC | STCP-1 | | CCR6 |
| I-TAC | TARC | TECK | | CCR7 |
| LIX | PARC | EST | | CCR8 |
| SCYB9 | LARC | MDC | | CXCR1 |
| B cell- | EBI 1 | Eotaxin | | CXCR2 |
| attracting | HCC-1 | | | CXCR3 |
| chemokine 1 | HCC-4 | | | CXCR4 |
| | | | | CXCR5 |
| | | | | CX₃CR |

TABLE 4
Interleukins and their receptors

| | | | | |
|---|---|---|---|---|
| G-CSF | IL-2 Rα | IL-8 | IL-16 | TGF-β1 |
| G-CSF R | IL-2 Rβ | IL-9 | IL-17 | TGF-β1,2 |
| GM-CSF | IL-2 Rγ | IL-9 R | IL-18 | TGF-β2 |
| IFN-γ | IL-3 | IL-10 | PDGF | TGF-β3 |
| IGF-I | IL-3 Rα | IL-10 R | PDGF A Chain | TGF-β5 |
| IGF-I R | IL-4 | IL-11 | PDGF-AA | LAP TGF-β1 |
| IGF-II | IL-4 R | IL-11 R | PDGF-AB | Latent TGF-β1 |
| IL-1α | IL-5 | IL-12 | PDGF B Chain | TGF-β b.p.1 |
| IL-1β | IL-5 Rα | IL-12 p40 | PDGF-BB | TGF-β RII |
| IL-1 RI | IL-6 | IL-12 p70 | PDGF Rα | TGF-β RIII |
| IL-1 RII | IL-6 R | IL-13 | PDGF Rβ | |
| IL-1rα | IL-7 | IL-13 Rα | TGF-α | |
| IL-2 | IL-7 R | IL-15 | TGF-β | |

TABLE 5
Human Non-CD Cellular Antigens

| Antigen | | | | | |
|---|---|---|---|---|---|
| Name | Other Name | MW | Structure | Distribution | Function |
| 4-1 BB | CD137L | | TNFSF | B^{act}, DC, | T costimulation |
| Ligand | | | | carcinoma cell | |
| | | | | lines | |
| AID | | | RNA-editing | B^{act}, Germinal | Activation-Induced |
| | | | deaminase | Center B | Deaminase, Ig class |
| | | | family | | switch recombination |
| AITR | TNFRSF18, | | | Treg, T^{act} | costimulation |
| | GITR | | | | |
| AITRL | TNFSF18, | | | APC | |
| | TL6, GITRL | | | | |
| B7 family | see CD273- | | | | |
| | 276 | | | | |
| B7-H4 | B7-S1, B7x | | B7 family | | may interact with BTLA |
| | | | | | (?), inhibition |
| BAMBI | TGFBR | 29 kD | TGFBR | carcinoma cell | pseudoreceptor for TGF-β |
| | | | | | (short cytoplasmic |
| | | | | | domain), growth inhibition |
| BCMA | see CD269 | | | | |
| BLyS | see CD257 | | | | |
| BR3 | see CD268 | | | | |
| BTLA | see CD272 | | | | |
| CCR7 | see CD197 | | | | |
| c-Met | HGFR/SFR | 190 kD | heterodimer, | epith, | tumor growth/metastasis, |
| | | | PTK | hematopoietic | Hepatocyte Growth |
| | | | | progenitors, | Factor/Scatter Factor |
| | | | | early | receptor, T development, |
| | | | | thymocytes | hematopoiesis |
| CMKLR1 | chemokine- | 42 kD | GPCR 7TM, | pDC (CD123+), | binds chemerin, pDC |
| | like receptor | | chemokine | in vitro derived | recruitment, bone |
| | 1 | | receptor | moDC | development |
| DcR3 | TR6, | | Soluble | tumors | Fas decoy receptor, tumor |
| | TNFRSF6B | | | | evasion |
| DEC-205 | see CD205 | | | | |
| DR3 | TRAMP, | | TNFRSF | T^{act}, leukocytes | lymphocyte homeostasis |
| | Apo-3, WSL- | | | | |
| | 1, LARD, TR3 | | | | |
| DR6 | TR7 | | TNFRSF | | death, Th2 response |
| FcεRIα | high-affinity | | tetramer | mast cells, | triggers IgE-mediated |
| | IgE receptor | | complex | basophils | allergic reactions |
| Foxp3 | SCURFIN | 50 kD | Fox family | T subsets | transcription factor, |
| | | | forkhead | (CD4+/CD25+ subset and | upregulated in T regs |
| | | | | CD8+ subset) | |
| Granzyme | Granzyme- | 30 kD | Peptidase | Cytotoxic T, NK | target cell apoptotic lysis, |
| B | 2, CTLA-1 | | S1 family | | cell-mediated immune |
| | | | | | responses |
| HLA-ABC | | 45, 11- | | nucleated cells | cell-mediated immune |
| | | 12 kD | | | response & tumor |
| | | | | | surveillance |
| HLA-DR | | | | APC, T^{act} | presentation of peptides to |
| | | | | | CD4+ T lymphocytes |
| HVEM | TNFRSF14, | 60 kD | TNFRSF | broad | receptor for LIGHT, LT-α, |
| | TR2 | | | expression | BTLA, Herpes Simplex |
| | | | | | Virus, lymphocyte |
| | | | | | activation |
| ICOS | see CD278 | | | | |
| ICOSL | see CD275 | | | | |
| IL-15Rα | | | | mono^{act} | binds to IL-15, w/IL-2RB |
| | | | | | and common γ, IL-15 |
| | | | | | trans-presentation |
| Integrin β5 | | 100 kD | | carcinoma cell | w/αv subunit, |
| | | | | lines, fibroblast | vitronectin receptor |
| | | | | lines | |
| MD-2 | | 30 KD | | w/TLR4 | |
| | | | | distribution and | |
| | | | | LPS recognition | |
| MICA/MICB | | 70 kD | MHC Class | intestinal epith, | unregulated on epith after |
| | | | I-related | some tumors | shock, NKG2D receptors |
| | | | proteins | | |
| Nanog | | 34 kD | | ES cells | transcription factor, self |
| | | | | | renewal of ES cells |
| NKG2D | see CD314 | | | | |
| NOD2 | CARD-15, | | | monocytes, | |
| | IBD1 | | | intracellular | |
| Notch-1 | Lin-12, | | | developing | cell-cell interaction, cell |
| | Tan 1 | | | embryo, variety | fate determination |
| | | | | of adult tissues | |
| OPG | TRAIL- R5, | | | binds TRAIL? | bone resorption |
| | TR1, | | | | |
| | TNFRSF11B | | | | |
| OX-40 | see CD134 | | | | |
| OX-40 | see CD252 | | | | |
| Ligand | | | | | |
| p38 | | 38 kD | SAP/MAP | NK, CD8+ T | role in cytolytic activity |
| | | | kinase | subset, | |
| | | | | upregulated on | |
| | | | | CD8+ T | |
| PD-1 | see CD279 | | | | |
| PD-L1 | see CD274 | | | | |
| PD-L2 | see CD273 | | | | |
| Perforin | | 70- | | CTL, NK | cytolytic protein |
| | | 75 kD | | | |
| RP105 | see CD180 | | | | |
| RANK | see CD265 | | | | |
| RANKL | see CD254 | | | | |
| SAP | SLAM- | 14 kD | adaptor | T, NK | negatively regulates |
| | associated | | protein | | SLAM- family receptors |
| | protein | | | | |
| SLP-76 | | 76 kD | | T, B^{low} | T cell receptor mediated |
| | | | | | signaling |
| SSEA-1 | stage- | | | ES cells, | down regulated by |
| | specific | | | embryonic | differentiation |
| | embryonic | | | carcinomas, | |
| | antigen-1 | | | germ cells | |
| SSEA-3 | stage- | | | | |
| | specific | | | | |
| | embryonic | | | | |
| | antigen-3 | | | | |
| SSEA-4 | stage- | | | | |
| | specific | | | | |
| | embryonic | | | | |
| | antigen-4 | | | | |
| Stro-1 | | | | BM stroma, | surface marker for |
| | | | | erythroid | immature mesenchymal |
| | | | | progenitors | cells |
| TACI | see CD267 | | | | |
| T-bet | | | | Th1 cells | transcription factor, T |
| | | | | | development/differentiation |
| TCL1 | | | | B cell tumors, | intracellular, lymphoid |
| | | | | lymphoid | proto-oncogene |
| | | | | lineages in a | |
| | | | | developmentally | |
| | | | | controlled | |
| | | | | manner, pDC | |
| TCR αβ | | | | peripheral T | antigen recognition |
| | | | | subset | |
| TCR γδ | | | | T subset | antigen recognition |
| TLR1- | see CD281- | | | | |
| TLR4 | CD284 | | | | |
| TLR5 | TIL3 | | TLR family | mRNA: | interacts w/microbial |
| | | | | leukocytes, | lipoproteins, NF-κB, |
| | | | | prostate, ovary, | responds to Salmonella |
| | | | | liver, lung | |
| TLR6 | | | TLR family | mRNA: | interacts w/microbial |
| | | | | leukocytes, | lipoproteins, protein |
| | | | | ovary, lung | sequence similar to |
| | | | | | hTLR1; regulates TLR2 |
| | | | | | response |
| TLR7 | | | TLR family | mRNA: spleen, | |
| | | | | placenta, lung; | |
| | | | | upregulated on | |
| | | | | mac | |
| TLR8 | | | TLR family | mRNA: | |
| | | | | leukocytes, lung | |
| TLR9 | see CD289 | | | | |
| TLR10 | | | TLR family | mRNA: | most closely related |
| | | | | lymphoid | to TLR1 and TLR6 |
| | | | | tissues | |
| TNFRI | see CD120a | | | | |
| TRAIL | see CD253 | | | | |
| TSLPR | | 50 kD | heterodimer | monocytes, DC, | binds TSLP (Thymic |
| | | | with IL- | B | Stromal Lymphopoietin) |
| | | | 7Rα/CD127 | | to activate DC |
| TWEAK | TNFSF12, | | TNFSF | activated mono | death |
| | APO3L | | | | |
| TWEAK | see CD266 | | | | |
| Receptor | | | | | |
| ULBPs | | | MHC class | tumors | NKG2D receptors, NK |
| | | | I-related | | activation |
| | | | protiens, | | |
| | | | GPI-linked | | |
| ZAP-70 | TCRζ- | 70 kD | Syk family | Intracellular T, | TCR signaling & |
| | associated | | | NK | development |
| | kinase | | | | |

It should be appreciated that in the above tables 1 through 5, an indicated gene means the gene and all currently known variants thereof, including the different mRNA transcripts to which the gene and its variants can give rise, and any further gene variants which may be elucidated. In general, however, such variants will have significant homology (sequence identity) to a sequence of a table above, e.g. a variant will have at least about 70 percent homology (sequence identity) to a sequence of the above tables 1-5, more typically at least about 75, 80, 85, 90, 95, 97, 98 or 99 homology (sequence identity) to a sequence of the above tables 1-5. Homology of a variant can be determined by any of a number of standard techniques such as a BLAST program.

Sequences for the genes listed in Tables 1-5 can be found in GenBank (www.ncbi.nlm.nih.gov/). The gene sequences may be genomic, cDNA or mRNA sequences. Preferred sequences are mammalian genes comprising the complete coding region and 5′ untranslated sequences. Particularly preferred are human cDNA sequences.

The methods of the invention can be used to screen for siRNA polynucleotides that inhibit the functional expression of one or more genes that modulate immune related molecule expression. For example, the CD-18 family of molecules is important in cellular adhesion. CD137, CD128, CTLA and ligands thereof are important in T cell co-stimulation. Through the process of adhesion, lymphocytes are capable of continually monitoring an animal for the presence of foreign antigens. Although these processes are normally desirable, they are also the cause of organ transplant rejection, tissue graft rejection and many autoimmune diseases. Hence, siRNA's capable of attenuating or inhibiting cellular adhesion would be highly desirable in recipients of organ transplants (for example, kidney transplants), tissue grafts, or for autoimmune patients.

In a preferred embodiment, the aptamers are specific to human non-CD antigens exemplified in table 5. However, aptamer specificities are not limited to the examples in Table 5 and can be any molecule the user wishes to target.

In another preferred embodiment, siRNA oligonucleotides modulate the expression of MHC molecules involved in immune responses. For example, Class I and Class II molecules of the MHC.

In another preferred embodiment, siRNA's are designed to target suppressor molecules that suppress the expression of gene that is not suppressed in a normal individual. For example, molecules involved in modulating a T cell response, such as for example, CD137, CTLA4, CD28, CD3, ligands, variants, mutants and fragments thereof, suppressor molecules which inhibit cell-cycle dependent genes, inhibition of p53 mRNA, inhibition of mRNA transcribed by genes coding for cell surface molecules (see tables 1-5), inhibition of caspases involved in apoptosis and the like.

The methods of the present invention can also be used to regulate the expression of a specific allele. Alleles are polymorphic variants of a gene that occupy the same chromosomal locus. The methods of the present invention allow for regulation of one or more specific alleles of a gene or a family of genes. In this embodiment, the sequence of the RNAi can be prepared such that one or more particular alleles of a gene or a family of genes are regulated, while other additional alleles of the same gene or family of genes are not regulated.

### Pharmaceutical Compositions

The invention also includes pharmaceutical compositions containing nucleic acid conjugates. In some embodiments, the compositions are suitable for internal use and include an effective amount of a pharmacologically active conjugate of the invention, alone or in combination, with one or more pharmaceutically acceptable carriers. The conjugates are especially useful in that they have very low, if any toxicity.

Compositions of the invention can be used to treat, prevent, diagnose or image a pathology, such as a disease or disorder, or alleviate the symptoms of such disease or disorder in a patient. For example, compositions of the invention can be used to treat, prevent, diagnose or image a pathology associated with inflammation. Compositions of the invention are useful for administration to a subject suffering from, or predisposed to, a disease or disorder which is related to or derived from a target to which the aptamers specifically bind or to the polynucleotides which the aptamer-delivered RNAi's are targeted to.

Compositions of the invention can be used in a method for treating a patient having a pathology, e.g. cancer. The method involves administering to the patient a composition comprising aptamers-RNAi's that bind a target (e.g., a protein), so that the RNAi is specifically delivered to a target cell of choice and altering the biological function of the target, thereby treating the pathology.

The patient having a pathology, e.g. the patient treated by the methods of this invention can be a mammal, or more particularly, a human.

In practice, the conjugate, aptamer-RNAi's, are administered in amounts which will be sufficient to exert their desired biological activity.

One aspect of the invention comprises a pharmaceutical composition of the invention in combination with other treatments for inflammatory and autoimmune diseases, cancer, and other related disorders. The pharmaceutical compositions of the invention may contain, for example, more than one aptamer-RNAi. In some examples, a pharmaceutical composition of the invention, containing one or more compounds of the invention, is administered in combination with another useful composition such as an anti-inflammatory agent, an immunostimulator, a chemotherapeutic agent, an antiviral agent, or the like. Furthermore, the compositions of the invention may be administered in combination with a cytotoxic, cytostatic, or chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

Combination therapy (or “co-therapy”) includes the administration of an aptamer-RNAi conjugate of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

Combination therapy may, but generally is not, intended to encompass the administration of two or more of these therapeutic agents as part of separate monotherapy regimens that incidentally and arbitrarily result in the combinations of the present invention. Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical unless noted otherwise. Combination therapy also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Therapeutic or pharmacological compositions of the present invention will generally comprise an effective amount of the active component(s) of the therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

For any aptamer-RNAi used in the methods of the invention, the therapeutically effective amount or dose can be estimated initially from activity assays in cell cultures and/or animals. For example, a dose can be formulated in animal models to achieve a circulating concentration range that includes the IC₅₀ as determined by activity assays (e.g., the concentration of the test compound, which achieves a half-maximal inhibition of the proliferation activity). Such information can be used to more accurately determine useful doses in humans.

Toxicity and therapeutic efficacy of the peptides described herein can be determined by standard pharmaceutical procedures in experimental animals, e.g., by determining the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1). Dosage amount and interval may be adjusted individually to provide plasma levels of the active moiety which are sufficient to maintain therapeutic effects, termed the minimal effective concentration (MEC). The MEC will vary for each preparation, but can be estimated from in vitro and/or in vivo data, e.g., the concentration necessary to achieve 50-90% inhibition of a proliferation of certain cells may be ascertained using the assays described herein. Dosages necessary to achieve the MEC will depend on individual characteristics and route of administration. HPLC assays or bioassays can be used to determine plasma concentrations. Dosage intervals can also be determined using the MEC value. Preparations should be administered using a regimen, which maintains plasma levels above the MEC for 10-90% of the time, preferable between 30-90% and most preferably 50-90%. Depending on the severity and responsiveness of the condition to be treated, dosing can also be a single administration of a slow release composition described hereinabove, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, etc.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or other known methods.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

For instance, for oral administration in the form of a tablet or capsule (e.g., a gelatin capsule), the active drug component can be combined with an oral, non-toxic, pharmaceutically acceptable inert carrier such as ethanol, glycerol, water and the like. Moreover, when desired or necessary, suitable binders, lubricants, disintegrating agents, and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes, and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol, and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

The compositions of the invention can also be administered in such oral dosage forms as timed release and sustained release tablets or capsules, pills, powders, granules, elixirs, tinctures, suspensions, syrups and emulsions. Suppositories are advantageously prepared from fatty emulsions or suspensions.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

The compositions of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compositions for the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would typically range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like. The active compound defined above, may be also formulated as suppositories, using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, and triethanolamine oleate. The dosage regimen utilizing the aptamer-RNAi's is selected in accordance with a variety of factors including type, species, age, weight, sex and medical condition of the patient; the severity of the condition to be treated; the route of administration; the renal and hepatic function of the patient; and the particular aptamer or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 7500 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Infused dosages, intranasal dosages and transdermal dosages will range between 0.05 to 7500 mg/day. Subcutaneous, intravenous and intraperitoneal dosages will range between 0.05 to 3800 mg/day. Effective plasma levels of the compounds of the present invention range from 0.002 mg/mL to 50 mg/mL. Compounds of the present invention may be administered in a single daily dose, or the total daily dosage may be administered in divided doses of two, three or four times daily.

### Other Embodiments

The foregoing paragraphs have described a preferred embodiment in which aptamers, RNAi's and aptamer-RNAi conjugates are synthesized. As those skilled in the art will readily appreciate, RNAi can also be produced through intramolecular hybridization of complementary regions within a single RNA molecule. An expression unit for synthesis of such a molecule comprises the following elements, positioned from left to right: 1. A DNA region comprising a viral enhancer; 2. A DNA region comprising an immediate early or early viral promoter oriented in a 5′ to 3′ direction so that a DNA segment inserted into the region of part 4 is transcribed; 3. A DNA region into which a DNA segment can be inserted. Preferably this region contains at least one restriction enzyme site; 4. A DNA region comprising a transcriptional terminator arranged in a 5′ to 3′ orientation so that a transcript synthesized in a left to right direction from the promoter of part 2 is terminated.

### Kits

In yet another aspect, the invention provides kits for targeting nucleic acid sequences of cells and molecules associated with modulation of the immune response in the treatment of diseases such as, for example, infectious disease organisms, cancer, autoimmune diseases and the like. For example, the kits can be used to target any desired nucleic sequence and as such, have many applications.

In one embodiment, a kit comprises: (a) an aptamer-RNAi that targets a desired cell and nucleic acid sequence, and (b) instructions to administer to cells or an individual a therapeutically effective amount of aptamer-RNAi. In some embodiments, the kit may comprise pharmaceutically acceptable salts or solutions for administering the aptamer-RNAi. Optionally, the kit can further comprise instructions for suitable operational parameters in the form of a label or a separate insert. For example, the kit may have standard instructions informing a physician or laboratory technician to prepare a dose of aptamer-RNAi.

Optionally, the kit may further comprise a standard or control information so that a patient sample can be compared with the control information standard to determine if the test amount of an aptamer-RNAi is a therapeutic amount consistent with for example, a shrinking of a tumor or decrease in viral load in a patient.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Embodiments of the invention may be practiced without the theoretical aspects presented. Moreover, the theoretical aspects are presented with the understanding that Applicants do not seek to be bound by the theory presented.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### Materials and Methods

### In Vitro Transcriptions

For 250 ml transcription reactions: 50 ml 5_T7 RNAP buffer optimized for 2′F transcriptions (20% wt/vol PEG 8000, 200 mM Tris-HCl pH 8.0, 60 mM MgCl₂, 5 mM spermidine HCl, 0.01% wt/vol Triton X-100, 25 mM DTT), 25 ml 10_{—}2′F-dNTPs (30 mM 2′F-CTP, 30 mM 2′F-UTP, 10 mM 2′OH-ATP, 10 mM 2′OH-GTP), 2 ml IPPI (Roche), 300 pmoles aptamer-siRNA chimera PCR template, 3 ml T7(Y639F) polymerase, bring up to 250 ml with milliQ H₂O.

The DNA templates for the transcriptions of the aptamer were generated with PCR using the library 5′ oligonucleotide and either of 2 3′ oligonucleotides: M12-23 CTLA-4 (5′-TGCTATATCCTTATGCTGCTTGGGGGGATCCAGTACT (SEQ ID NO: 1)) or M12-23 con (5′-CTGCAGGATGTTCTCATGCTTGGGGGGATCCAGTACT-3′ (SEQ ID NO: 2)); bold sequence denotes the portion that encodes the siRNA region. Templates for the PCRs were plasmid clones of either the M12-23 or mutM12-23 sequences. To prepare chimeras, 10 μM gel-purified sense RNA was combined with 20 μM of the appropriate antisense RNA in DPBS, heated to 65° C. for 5 min and then cool down to 37° C. for 10 min. The volume was reduced by centrifugal filtration (Centricon YM-30; Millipore).

### Predicting RNA Secondary Structure

RNA Structure Program version 4.1 (rna.urmc.rochester.edu/rnastructure.html) was used to predict these secondary structures of aptamer-siRNA chimera. The most stable structures with the lowest free energies for each RNA oligonucleotide were compared.

### Purification of T Cells

CD8⁺T cells were purified from the spleens and lymph nodes of BALB/c mice with a MACS Negative selection kit (Miltenyi Biotech). After lysing the red blood cells in NH₄Cl and removing adherent cells, the procedure outlined in the manufacturer's instructions was followed closely. At the completion of the purification, cells were pelleted by centrifugation, resuspended in Dulbecco PBS (DPBS; without Ca²⁺ and Mg²⁺) plus 5% FBS and counted. These preparations were occasionally assayed for purity with an anti-CD8 Ab and flow cytometry and consistently found to include greater than 95% CD8⁺ cells.

### CFSE Labeling and Cell Culture

Purified CD8⁺ mouse T cells were labeled with 2 μM CFSE (Invitrogen) for 5 minutes at room temperature in DPBS (without Ca²⁺ and Mg²⁺) plus 5% FBS with a cellular concentration of 10⁶ cells/ml. Cells were then washed twice with DPBS (without Ca²⁺ and Mg²⁺) plus 2% FBS and then once with complete T cell culture media (see below). Purified, CFSE-labeled or unlabeled cells were plated at 5×10⁵ cells/well, 200 μl/well in 96-well round-bottomed culture dishes in complete T cell media (RPMI 1640 supplemented with 10% FBS, 1 mM sodium pyruvate, essential and nonessential amino acids, 100 U/ml penicillin, 100 μg/ml streptomycin, 55 μM β-mercaptoethanol, and 20 mM HEPES). Plates were coated with CD3e at 0.5 μg/ml. At 16 hours after plating, cell were removed into another 96-well plate and new complete T cell media with anti-4-1BB Aptamer-siRNA chimeras in solution (200 nM) were added to the cells. After 48 hours of incubation the cell were plated again in a 96 well plate coated with anti-CD3e antibodies 0.1 μg/ml and fresh T cell media with anti CD28 3 μg/ml was added to the cells. 64 hours later CFSE was measured by flow cytometry.

### IL-2 ELISA

Mouse CD8⁺T cells were purified as described above. Plates were coated with CD3e at 0.5 μg/ml. At 16 hours after plating, cell were removed into another 96-well plate and new complete T cell media with anti-4-1BB. Aptamer-siRNA chimeras in solution (200 nM) were added to the cells. After 48 hours of incubation the cell were plated again in a 96 well plate coated with anti-CD3e Ab and fresh T cell media with 10³ adherent splenocytes was added to the cells in each well At 24 hours after plating, the supernatants were removed and assayed with a mouse IL-2 ELISA kit (BD). Three wells of cells were assayed for each condition.

### RT-PCR

Mouse CD8⁺T cells were purified as described above. Plates were coated with CD3e at 0.5 μg/ml. At 16 hours after plating, cell were removed into another 96-well plate and new complete T cell media with anti-4-1BB Aptamers-siRNA chimeras in solution (200 nM) was added to the cells. After 48 hours total RNA was extracted using the Qiagen kit. Retrotranscription was set up with a MultiScript kit (Applied Biosystems) 20 μl final volume, 1 μg total RNA was used for each reaction. The PCR was done with equal amount of cDNA, using 3′ primer AAAATGCCCCCAACAGAGCC (SEQ ID NO: 3) and as 5′ primer CCACCAGCAAATACACAACAGCAC (SEQ ID NO: 4) for CTLA4 PCR, and the primers for the actin: 3′ primer CCACACTGTGCCCATCTACG (SEQ ID NO: 5), 5′ primer GATCTTCATGGTGCTAGGAGC (SEQ ID NO: 6).

Design and Characterization of a 4-1BB Aptamer-CTLA-4 siRNA

A fusion between a 4-1BB aptamer (using a monomeric form which does not induce costimulation) and a siRNA against CTLA-4 was generated, whereby the siRNA was conjugated to the 3′ end of the aptamer, using a double stranded linker. Incubation of polyclonally activated CD8⁺T cells with the 4-1BB aptamer-CTLA-4 siRNA ODN, but not with control ODNs containing either a mutant non-binding aptamer or a control siRNA, led to the downregulation of CTLA-4 expression and a concomitant enhanced proliferation of the T cells or IL-2 secretion.

Development of a Dual Function 4-1 BB Aptamer-eTLA-4 siRNA ODN

Murine studies have shown that two or more antibodies targeting complementary pathways can exert synergistic or additive effects in promoting protective antitumor immunity. For example, treatment with anti-4-1 BB antibody and anti-B7H1 antibody or a combination of anti-DRS, anti-4-1 BB and anti-CD40 antibodies exhibited remarkable antitumor effects. Co-administration of blocking anti-CTLA-4 antibody together with an agonistic anti-4-1BB antibody not only enhanced antitumor immunity but also attenuated CTLA-4 antibody-induced autoimmunity, most likely reflecting the suppressive effects of 4-1 BB co-stimulation on autoimmune sequalea.

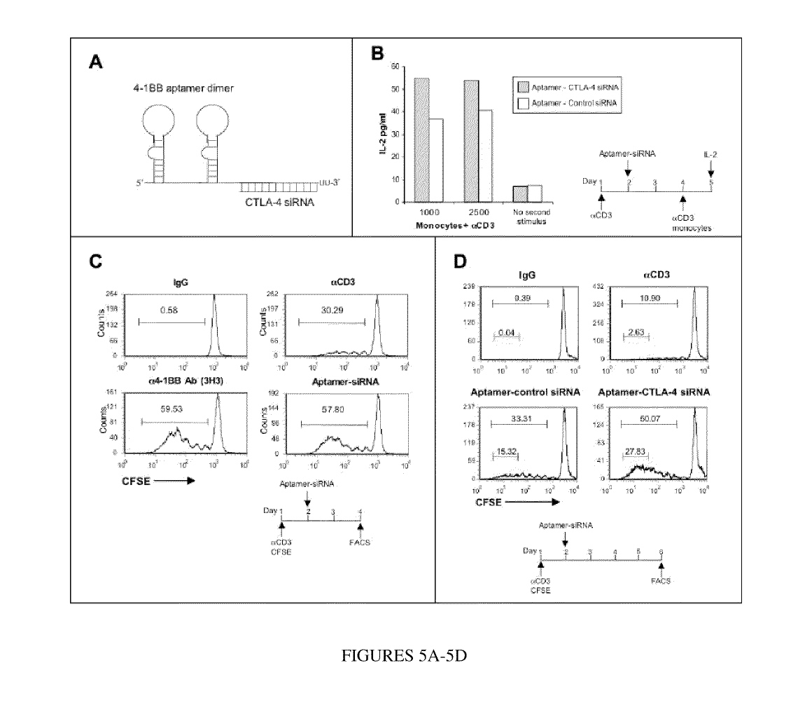

To obtain evidence that 4-1 BB co-stimulation and CTLA-4 inhibition can be incorporated into one ODN, a chimeric ODN was generated in which one of the CTLA-4 siRNA is conjugated to a dimeric form of the 4-1 BB aptamer as shown in FIG. 5A. This was achieved by in effect adding a second monomeric 4-1 BB aptamer to the 5′ end of the 4-1 BB aptamer-CTLA-4 siRNA chimera. First, the function of each component was determined separately. FIG. 5B shows that CTLA-4 siRNA, but not control siRNA, conjugated to the 4-1 BB aptamer dimer enhances IL-2 secretion by the activated T cells, and FIG. 5C shows that the dimeric 4-1 BB aptamer-control siRNA chimera enhances the proliferation of suboptimally activated CD8⁺T cells to an extent comparable to that of 4-1 BB antibodies.

To determine if the aptamer and siRNA components of the chimeric ODN can synergize in co-stimulating activated T cells, the proliferative capacity of sub-optimally stimulated CD8⁺T cells was measured, as shown in FIG. 5D. Under suboptimal stimulation 10.90% of the CD8⁺T cells proliferated, 2.63% extensively (αCD3 panel). When cells were also co-incubated with a 4-1 BB dimer aptamer—control siRNA, proliferation was enhanced; 33.1% of cells proliferated, 15.32% extensively. This enhanced proliferation represented the effect of 4-1 BB co-stimulation. When cells were incubated with the 4-1 BB aptamer dimer—CTLA-4 siRNA, proliferation was further enhanced about two-fold, 50.07% cells proliferating, 27.83% extensively, reflecting the contribution of CTLA-4 inhibition. When cells were incubated with 4-1BB aptamer dimer-CTLA-4 siRNA in the absence of anti-CD3 antibody (IgG panel) no proliferation was observed, all but excluding off target effects. The data shown in FIG. 5D evidence that both 4-1 BB costimulation and CTLA-4 blockade contributed to enhanced proliferation of the polyclonally activated CD8⁺T cells.

Significance: Instead of using two separate agents—hard-to-access antibodies—this dual-function aptamer-siRNA incorporates two functionalities in one oligonucleotide (with all the advantages of oligonucleotide versus protein-based therapeutics) which is also targeted to the desired cell.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the Disclosure is provided to allow the reader to quickly ascertain the nature of the technical disclosure. It is submitted with the understanding that it will not be used to interpret or limit the scope or meaning of the following claims.

### REFERENCES

1. Pastan, I., R. Hassan, D. J. Fitzgerald, and R. J. Kreitman. 2006. Immunotoxin therapy of cancer. Nat Rev Cancer 6:559-565.
2. Pardoll, D., and J. Allison. 2004. Cancer immunotherapy: breaking the barriers to harvest the crop. Nat Med 10:887-892.
3. Uno, T., K. Takeda, Y. Kojima, H. Yoshizawa, H. Akiba, R. S. Mittler, F. Gejyo, K. Okumura, H. Yagita, and M. J. Smyth. 2006. Eradication of established tumors in mice by a combination antibody-based therapy. Nat Med 12:693-698.
4. Zitvogel, L., A. Tesniere, and G. Kroemer. 2006. Cancer despite immunosurveillance: immunoselection and immunosubversion. Nat Rev Immunol 6:715-727.
5. Galon, J., W. H. Fridman, and F. Pages. 2007. The adaptive immunologic microenvironment in colorectal cancer: a novel perspective. Cancer Res 67:1883-1886.
6. Nimjee, S. M., C. P. Rusconi, and B. A. Sullenger. 2005. Aptamers: an emerging class of therapeutics. Annu Rev Med 56:555-583.
7. McNamara, J. O., 2nd, E. R. Andrechek, Y. Wang, K. D. Viles, R. E. Rempel, E. Gilboa, B. A. Sullenger, and P. H. Giangrande. 2006. Cell type-specific delivery of siRNAs with aptamer-siRNA chimeras. Nat Biotechnol 24:1005-1015.
8. Gorelik, L., and R. A. Flavell. 2001. Immune-mediated eradication of tumors through the blockade of transforming growth factor-beta signaling in T cells. Nat Med 7:1118-1122.

What is claimed:

1. A composition for modulating immune cells comprising an aptamer-interference RNA (RNAi) fusion molecule wherein said molecule is targeted to cells and cellular molecules associated with regulation of an immune response and comprises at least one aptamer specific for at least one molecule.

2. The composition of claim 1, wherein the interference RNA comprising at least one of a short interfering RNA (siRNA); a micro, interfering RNA (miRNA); a small, temporal RNA (stRNA); or a short, hairpin RNA (shRNA).

3. The composition of claim 1, wherein the immune cells comprise T cells (T lymphocytes), B cells (B lymphocytes), antigen presenting cells, dendritic cells, monocytes, macrophages, myeloid suppressor cells, natural killer (NK) cells, cytotoxic T lymphocytes (CTLs), CTL lines, CTL clones, CTLs from tumor, inflammatory, or other infiltrates and subsets thereof.

4. The composition of claim 3, wherein the aptamer is specific for T lymphocytes and subsets thereof.

5. The composition of claim 4, wherein the aptamer is specific for CD8⁺T lymphocytes and markers thereof.

6. The composition of claim 1, wherein the aptamer is specific for T regulatory cells.

7. The composition of claim 1, wherein the aptamer is specific for molecules comprising 4-1BB (CD137), OX40, CD3, CD28, HLA-ABC, HLA-DR, T Cell receptor αβ (TCRαβ), T Cell receptor γδ (TCRγδ), T cell receptor ζ (TCRζ), TNF receptor, Cd11c, CD1-339, B7, mannose receptor, or DEC205, variants, mutants, ligands, alleles and fragments thereof.

8. The composition of claim 1, wherein the interference RNA (RNAi) is specific for polynucleotides comprising TGFβ receptor, polynucleotides associated with TGFβ signaling, purinergic receptors, CTLA-4, PTEN, Csk, Cb1-b, cytokines, SOCS1, GILT, GILZ, A20 or Bax/Bak.

9. The composition of claim 8 wherein the interference RNA (RNAi) is specific for polynucleotides associated with TGFβ signaling.

10. The composition of claim 1 wherein the RNAi targets TGFβ in activated T lymphocytes.

11. The composition of claim 1, wherein the aptamer-RNA interference fusion molecule comprises at least one oligonucleotide as set froth in SEQ ID NOS: 1-6.

12. A method of modulating an immune response in patient comprising:
constructing an aptamer and/or targeting agent and interference RNA fusion molecule wherein the aptamer and/or targeting agent is specific for an immune effector cell and the interference RNA is specific for a molecule associated with attenuation or suppression of the immune effector cell;
administering the fusion molecule in a therapeutically effective amount to the patient; and,
modulating the immune response.

13. The method of claim 12, wherein the aptamer and or targeting agent are specific for an activated CD8⁺T lymphocyte or CD8⁺T lymphocyte molecules thereof, and the interference RNA is specific for TGFβ, TGFβRII, variants, mutants and fragments thereof.

14. The method of claim 12, wherein an aptamer-interference RNA comprises at least one of an oligonucleotide as set forth in SEQ ID NOS: 1-6.

15. The method of claim 12, wherein the aptamer-interference RNA fusion molecule comprising: at least one aptamer specific for a desired cell marker for targeting the fusion molecule, and at least one interference RNA molecule specific for a desired polynucleotide.

16. The method of claim 12, wherein the aptamer-interference RNA fusion molecule comprises a linker molecule.

17. The method of claim 12, wherein the polynucleotide encoding the aptamer-interference RNA fusion molecule comprises one or more nucleotide substitutions.

18. The method of claim 17, wherein the nucleotide substitutions comprise at least one or combinations thereof, of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants and analogs thereof.

19. The method of claim 12, wherein the linker molecule comprises nucleotide, non-nucleotide, or mixed nucleotide/non-nucleotide linker joining the one or more aptamers to on or more interference RNA molecules.

20. The method of claim 19, wherein the one or more linker molecules comprising about 2 nucleotides length up to about 50 nucleotides in length.

21. The method of claim 19, wherein the non-nucleotide linker comprising abasic nucleotide, polyether, polyamine, polyamide, peptide, carbohydrate, lipid, polyhydrocarbon, or polymeric compounds having 1 or more monomeric units.

22. An aptamer-interference RNA molecule comprising at least one aptamer specific for a marker of a target cell and at least one interference RNA molecule specific for a desired polynucleotide of the target cell.

23. The aptamer-interference RNA molecule of claim 22, wherein the at least one aptamer is linked to the at least interference RNA by at least one linker molecule.

24. The aptamer-interference RNA molecule of claim 23, wherein the linker molecule comprises wherein the linker molecule comprises nucleotide, non-nucleotide, or mixed nucleotide/non-nucleotide linker joining the one or more aptamers to on or more interference RNA molecules.

25. The aptamer-interference RNA molecule of claim 23, wherein the one or more linker molecules comprising about 2 nucleotides length up to about 50 nucleotides in length.

26. The aptamer-interference RNA molecule of claim 23, wherein the non-nucleotide linker comprises abasic nucleotide, polyether, polyamine, polyamide, peptide, carbohydrate, lipid, polyhydrocarbon, or polymeric compounds having 1 or more monomeric units.

27. The aptamer-interference RNA molecule of claim 23, wherein the polynucleotide encoding the aptamer-interference RNA fusion molecule comprises one or more nucleotide substitutions.

28. The aptamer-interference RNA molecule of claim 27, wherein the nucleotide substitutions comprise at least one or combinations thereof, of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³—C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants and analogs thereof.

29. The aptamer-interference RNA molecule of claim 22, wherein the aptamer is specific for molecules comprising 4-1BB (CD137), OX40, CD3, CD28, or HLA-DR, CD11c, mannose receptor or DEC205variants, mutants, alleles and fragments thereof.

30. The aptamer-interference RNA molecule of claim 22, wherein the interference RNA (RNAi) is specific for polynucleotides comprising TGFβ receptor, polynucleotides associated with TGFβ signaling, purinergic receptors, CTLA-4, PTEN, Csk, Cb1-b, cytokines, SOCS1, GILT, GILZ, A20 or Bax/Bak.

31. The aptamer-interference RNA molecule of claim 22, wherein the aptamer is specific for 4-1BB (CD137), OX40, CD3, CD28, HLA-ABC, HLA-DR, T Cell receptor αβ (TCRαβ), T Cell receptor γδ (TCRγδ), T cell receptor ζ (TCRζ), TNF receptor, Cd11c, CD1-339, B7, mannose receptor, or DEC205, variants, mutants, ligands, alleles and fragments thereof.

32. The aptamer-interference RNA molecule of claim 22, wherein the interference RNA comprising at least one of a short interfering RNA (siRNA); a micro, interfering RNA (miRNA); a small, temporal RNA (stRNA); or a short, hairpin RNA (shRNA).

33. A composition for modulating immune cells comprising a targeting agent-interference RNA (RNAi) fusion molecule wherein said molecule is targeted to cells and cellular molecules associated with regulation of an immune response, comprising at least one targeting agent which specifically binds to at least one molecule.

34. The composition of claim 33, wherein the interference RNA comprising at least one of a short interfering RNA (siRNA); a micro, interfering RNA (miRNA); a small, temporal RNA (stRNA); or a short, hairpin RNA (shRNA).

35. The composition of claim 33, wherein the immune cells comprise T cells (T lymphocytes), B cells (B lymphocytes), antigen presenting cells, dendritic cells, monocytes, macrophages, myeloid suppressor cells, natural killer (NK) cells, cytotoxic T lymphocytes (CTLs), CTL lines, CTL clones, CTLs from tumor, inflammatory, or other infiltrates and subsets thereof.

36. The composition of claim 33, wherein the targeting agent comprising: aptamers, antibodies, integrins, receptors, ligands, peptides, or RGD based peptides.

37. The composition of claim 33, wherein the aptamer is specific for T lymphocytes and subsets thereof comprising: CD8⁺T lymphocytes or markers thereof.

38. The composition of claim 33, wherein the aptamer or targeting agents are specific for T regulatory cells.

39. The composition of claim 33, wherein the aptamer or targeting agents are specific for molecules comprising 4-1BB (CD137), OX40, CD3, CD28, HLA-ABC, HLA-DR, T Cell receptor αβ (TCRαβ), T Cell receptor γδ (TCRγδ), T cell receptor ζ (TCRζ), TNF receptor, Cd11c, CD1-339, B7, mannose receptor, or DEC205, variants, mutants, ligands, alleles and fragments thereof.

40. The composition of claim 33, wherein the interference RNA (RNAi) is specific for polynucleotides comprising TGFβ receptor, polynucleotides associated with TGFβ signaling, purinergic receptors, CTLA-4, PTEN, Csk, Cb1-b, cytokines, SOCS1, GILT, GILZ, A20 or Bax/Bak.

41. The composition of claim 40 wherein the interference RNA (RNAi) is specific for polynucleotides associated with TGFβ signaling.

42. The composition of claim 33, wherein the RNAi targets TGFβ in activated T lymphocytes.

43. The composition of claim 33, wherein two or more targeting agents specifically bind to different molecules or same molecules.

44. A method of treating tumors in vivo comprising:
administering to a patient in need thereof a therapeutically effective chimeric molecule which specifically binds to immune cells or cells in tumor vasculatures; and,
treating tumors in vivo.

45. The method of claim 44, wherein the chimeric molecule comprises one or more targeting agents with same or different specificities fused or linked to an interference RNA molecule.
